(19) United States
(12) Patent Application Publication
(10) Pub. No.: US 2024/0409883 A1
(43) Pub. Date: Dec. 12, 2024

(54) MANGANESE SCAVENGING LACTOBACILLI AND USES THEREOF

(71) Applicant: Chr. Hansen A/S, Hoersholm (DK)

(72) Inventors: Solvej Siedler, Hoersholm (DK); Martin Holm Rau, Hoersholm (DK); Elleke Bosma, Hoersholm (DK); Anna Boguta, Hoersholm (DK)

(73) Assignee: Chr. Hansen A/S, Hoersholm (DK)

(21) Appl. No.: 18/686,009

(22) PCT Filed: Aug. 26, 2022
(86) PCT No.: PCT/EP2022/073776

§ 371 Date: Feb. 23, 2024

(30) Foreign Application Priority Data
Aug. 26, 2021 (EP) 21193288.4

(51) Int. Cl. C12N 1/20; C12N 15/74

(52) U.S. Cl. CPC C12N 1/20; C12N 15/746; C12N 2800/101

(57) ABSTRACT

The present invention is in the field of microbiology. The present application provides novel lactobacilli with inactivated regulatory sequences for manganese transporter MntH1 as well as composition comprising the bacteria. Such bacteria can be used for manufacturing food products or other industrial applications. Furthermore, the present invention provides a method of improving manganese scavenging activity in lactobacilli.

### REFERENCE TO A SEQUENCE LISTING

This application contains a Sequence Listing in computer readable form which is incorporated herein by reference in its entirety. The contents of the electronic sequence listing was created on Jun. 6, 2024, named SQ.xml and has 75,123 bytes in size. This replaces the previously filed sequence listing.

### FIELD OF THE INVENTION

The present invention lies in the field of microbiology and relates to bacteria which have manganese uptake activity. The bacteria can be used for controlling spoilage or contamination of unwanted microorganisms in products. The invention also relates to fermented food products and preparations thereof using such bacteria.

### BACKGROUND OF THE INVENTION

A major problem in the food industry is spoilage by unwanted microorganisms. Yeasts and molds are highly efficient at causing foods to spoil and are a problem for most food manufacturers. Spoilage due to yeasts and molds is clearly visible as patches of mold or discoloration on the surface of the food product, allowing it to be disposed of prior to consumption. Yeasts tend to grow within food and drink matrices in planktonic form. They tend to ferment sugars and grow well under anaerobic conditions. In contrast, molds tend to grow on the surface of products in the shape of a visible mycelium made up of cells.

Premature microbial spoilage of dairy products, including fluid milk, cheese, and cultured products, is a primary contributor to dairy food waste. Microbial contamination may occur at various points throughout the production and processing continuum and includes organisms such as gram-negative bacteria (e.g., Pseudomonas), gram-positive bacteria (e.g., Paenibacillus) and a wide range of fungal organisms.

Besides spoilage, food contamination in food products is a constant challenge in the industry. For example, listeria contamination is relevant in some dairy products and ready-to-eat (RTE) foods, and may lead to severe illness, including severe sepsis, meningitis, or encephalitis, sometimes resulting in lifelong harm and even death. In dairy products, milk heat treatment is not always sufficient to guarantee the absence of Listeria monocytogenes. It is known that a lack of hygiene or sanitation during the post-pasteurization or post-processing steps would also lead to contamination. There is a constant need to control listeria growth in the food industry.

Manganese depletion has been reported as a mechanism in lactic acid bacteria (LAB) to delay the growth of spoilage contaminants in dairy products (Siedler et al. “Competitive exclusion is a major bioprotective mechanism of lactobacilli against fungal spoilage in fermented milk products.” Appl Environ Microbiol 86 (2020): e02312-19. and van Gijtenbeek, Lieke A., et al. “Lacticaseibacillus rhamnosus impedes growth of Listeria spp. in cottage cheese through manganese limitation.” Foods 10.6 (2021): 1353). Manganese (Mn) is an essential trace element that is a key cofactor in all kingdoms of life, making it important for the growth of bacteria, yeast and mold. Furthermore, low manganese concentrations can serve as limiting factor for listeria growth.

WO2019/202003 discloses fungal inhibition by using bacteria with manganese uptake activities. The two major manganese uptake systems in LAB are the NRAMP-type transporter MntH and the ABC transporter SitABC. While the ABC transporter is mainly active at neutral pH, the proton-driven symporter MntH is the major transport system under acidic condition. In particular, high expression of MntH contributes significantly to manganese uptake, which limited manganese availability for growth of other microorganisms (Siedler et al., 2000).

For economic and environmental reasons, there is a constant need for improved strategies which are effective for controlling microbial spoilage or contamination.

### SUMMARY OF THE INVENTION

The present application relates to inhibition of microbial growth by manganese depletion. The inventors have for the first time discovered that the Lactobacillus strains are able to scavenge manganese in the presence of higher manganese concentrations in the environment when the repressor mechanism for the transcription of manganese transporter MntH1 is disrupted.

Based on this, it is now possible to provide strains whose manganese uptake ability is improved. This makes them especially useful for applications in products having higher manganese level, since the uptake ability of the strains is improved.

As will be described in detail below, the manganese transport regulator MntR (also referred to as “MntR protein” or simply “MntR”) acts as repressor for the transcription of mntH1. The bacteria according to the present application are characterized by inactivated MntR protein and/or corresponding binding site for MntR. This may be provided by directly screening for bacteria with such features, or by mutating relevant genes in the wild type mother strain and selecting therefrom mutants with higher manganese scavenging activity.

In a first aspect, the present application provides a method of improving manganese scavenging activity in a lactobacillus strain, comprising:
providing one or more Lactobacillus strains which express the manganese transporter MntH1 as the mother strain,
obtaining one or more mutants from the mother strain, in which the manganese transport regulator MntR or its binding site upstream of mntH1 gene is inactivated, and
selecting from the obtained mutants one or more daughter Lactobacillus strains whose manganese scavenging activity is higher compared to the mother strain

The selecting step may be performed at in a suitable medium at a predetermined manganese concentration, such as 0.135 mg/L, 0.2 mg/L, 0.3 mg/L, 0.4 mg/L, 0.5 mg/L or 1.0 mg/L.

Preferably, the Lactobacillus strains belong to the species of L. salivarius, L. reuteri, L. brevis, L. kefiri, L. alimentarius, L. zeae, L. kimchicus, L. curvatus, L. sakei, L. casei, L. paracasei, L. rhamnosus, L. plantarum and L. fermentum. Most preferably, the Lactobacillus strains belong to the species of L. curvatus, L. sakei, L. casei, L. paracasei, L. rhamnosus, L. plantarum and L. fermentum.

In a further aspect, the present application provides Lactobacillus spp. comprising a manganese transporter MntH1, characterized in that the strain comprises inactivated manganese transporter regulator MntR and/or inactivated binding site for MntR upstream of mntH1.

Preferably, the Lactobacillus strains belong to the species of L. salivarius, L. reuteri, L. brevis, L. kefiri, L. alimentarius, L. zeae, L. kimchicus, L. curvatus, L. sakei, L. casei, L. paracasei, L. rhamnosus, L. plantarum and L. fermentum. Most preferably, the Lactobacillus strains belong to the species of L. curvatus, L. sakei, L. casei, L. paracasei, L. rhamnosus, L. plantarum and L. fermentum.

In another aspect, the present application provides a method of reducing free manganese in a product, preferably food or feed product, comprising the steps of:
selecting one or more manganese scavenging Lactobacillus strains comprising manganese transporter MntH1 and inactivated manganese transporter regulator MntR and/or inactivated binding site for MntR upstream of mntH1,
adding the Lactobacillus strain(s), preferably as a Direct Vat Set (DVS) culture composition, to the product, and thereby reducing free manganese.

The manganese scavenging activity of the Lactobacillus may lead to the inhibition or delay of the growth of unwanted microorganisms, such as yeast, mold and/or other bacteria such as listeria.

It is also preferred that the manganese in the product is reduced to a concentration of below 0.01 ppm, preferably below about 0.008 ppm, and more preferably below 0.006 ppm.

The present application additionally provides a composition of Lactobacillus strain(s) with improved manganese scavenging activity which can be in a frozen, dried or freeze-dried form, e.g. as a Direct Vat Set (DVS) culture, preferably with a concentration of at least 10⁶ colony forming unit/g (cfu/g), such as at least 10⁷, at least 10⁸, at least 10⁹ or at least 10¹⁰ cfu/g. The composition may further comprise further bacteria, such as lactic acid bacteria, including Streptococcus thermophilus and/or Lactobacillus delbrueckii subsp. bulgaricus.

In a further aspect, the present application provides the use of one or more Lactobacillus strain(s) with improved manganese scavenging activity to inhibit or delay fungal or listeria growth in food or feed products. Preferably, the use is carried out in the presence of glucose. The inventors have surprisingly found that the manganese uptake may be increased under such condition.

Glucose can be already present in the product applied. Alternatively, it may be supplemented by direct addition, or indirectly, for example, by adding at least one lactic acid bacteria strain(s) which is able to release glucose as metabolite. In preferred embodiments, the use is carried out in the presence of at least 0.2 g/L glucose, such at least 0.5 g/L glucose, such at least 1.0 g/L glucose, such at least 2.0 g/L glucose, such at least 3.0 g/L glucose, such at least 4.0 g/L glucose, such at least 5.0 g/L glucose.

The present invention also provides products, such as food product, feed products, cosmetic product, health care product or a pharmaceutical product, comprising the manganese scavenging Lactobacillus strain(s) described herein. Such products may be fermented food product, dairy product, dairy analogue product, meat product, meat analogue product or vegetable product or the like.

Throughout this disclosure, gene names are denoted with italicized letters, and the proteins associated with the genes are denoted in non-italicized letters with the first letter capitalized.

### BRIEF DESCRIPTION OF THE FIGURES

FIG. 1 depicts the measured pH in milk supplemented with the indicated manganese concentration after 24 hours of incubation at 37° C. in milk (FIG. 1A) and milk supplemented with 0.5% glucose (FIG. 1B) for the L. paracasei mother strain (black circles) and its mntR deletion mutant LpMntR (grey diamonds). Individual values and mean are shown of three biological independent experiments.

FIG. 2 depicts the red fluorescence measured in milk supplemented with the indicated manganese concentration after 24 hours of incubation at 37° C. in milk (FIG. 2A) and milk supplemented with 0.5% glucose (FIG. 2B) for the L. paracasei mother strain (black circles) and its mntR deletion mutant LpMntR (grey diamonds). Individual values and mean are shown of three biological independent experiments.

FIG. 3 depicts the results of yeast inhibition assay. After growth in milk supplemented with the indicated manganese concentration for 24 hours without glucose addition (FIG. 3A) or with glucose addition (FIG. 3B), the bioassay was performed in biological duplicates and about 20 CFUs of D. hansenii were introduced to the samples. After 5 days of incubation at 17 C a 1000-fold dilution was spotted on selective YGC plates. The pictures were taken after two days of incubation at room temperature. The results of two biological independent experiments are shown for each strain.

FIG. 4 depicts acidification of CHCC15860 (FIG. 4A) and LrMntR (FIG. 4B) in milk supplemented with the indicated manganese concentration during incubation at 37° C. in milk for 21 hours.

FIG. 5 depicts the results of yeast inhibition assay. After growth of the indicated strain in milk supplemented with the indicated manganese concentration for 24 hours, the bioassay was performed. For this, about 20 CFUs of D. hansenii were introduced to the samples. After 4 days of incubation at 17° C. a 100-fold dilution was spotted on selective YGC plates. The pictures were taken after two days of incubation at room temperature. No yeast growth indicating inhibition, while yeast growth indicates no inhibition by the bioprotective strain at the given conditions.

### DETAILED DESCRIPTION OF THE INVENTION

### Manganese Transporters

Transport systems for manganese are known and for example described in Kehres et al., “Emerging themes in manganese transport, biochemistry and pathogenesis in bacteria.” FEMS microbiology reviews 27.2-3 (2003): 263-290. Bacterial Mn²⁺ transporters include ABC transporter (for example SitABCD and YfeABCD) or proton-dependent Nramp-related transporters.

While the ABC transporter is mainly active at higher pH, the proton driven transporters are more active under acidic conditions. Proton driven transporters are thus particularly useful as manganese scavenging agents in fermented food or feed products.

MntH belongs to the metal ion (Mn²⁺-iron) transporter (Nramp) family designated as TC #2.A.55 in the transporter classification system given by the Transport Classification Database (M. Saier; U of CA, San Diego, Saier M H, Reddy V S, Tamang D G, Vastermark A. (2014)). The TC system is a classification system for transport proteins which is analogous to the Enzyme Commission (EC) system for classification of enzymes. The transporter classification (TC) system is an approved system of nomenclature for transport protein classification by the International Union of Biochemistry and Molecular Biology. TCDB is freely accessible at http://www.tcdb.org which provides several different methods for accessing the data, including step-by-step access to hierarchical classification, direct search by sequence or TC number and full-text searching. Different MntH homologues transporters have been described by Groot et al. “Genome-based in silico detection of putative manganese transport systems in Lactobacillus plantarum and their genetic analysis.” Microbiology 151.4 (2005): 1229-1238. The present invention relates to in particular bacteria which express the manganese transporter MntH1 and its transcriptional regulator.

### Manganese Transporter Regulator MntR

The manganese transport regulator MntR is a metalloprotein transcriptional regulator that is activated by Mn²⁺ to repress transcription of the manganese transporter. MntR controls intracellular Mn²⁺ levels by coordinating the transcription of importers and, depending on the organisms, the exporters. MntR forms a homodimer that, through binding of one Mn²⁺ ion per subunit, undergoes a conformational change, which increases affinity for its DNA binding sites.

The first studies on the function and regulation of Mn²⁺ metabolism focused on E. coli and S. typhimurium as model organisms. Subsequent investigations in B. subtilis and to some extent in streptococci and lactococci have been performed, for example, as described by Que et al. 2000 (“Manganese homeostasis in Bacillus subtilis is regulated by MntR, a bifunctional regulator related to the diphtheria toxin repressor family of proteins.” Molecular microbiology 35.6 (2000): 1454-1468).

In B. subtilis, it has been shown that a tight regulation is required to correctly balance the intracellular concentration of Mn, a trace element that is both essential and toxic. Bacillus subtilis mntR deletion mutants was observed to constitutively express both MntH and MntABC, which lead to Mn²⁺ intoxication (Huang, et al. “Bacillus subtilis MntR coordinates the transcriptional regulation of manganese uptake and efflux systems.” Molecular microbiology 103.2 (2017): 253-268).

Mn²⁺ uptake regulation has not been studied in lactic acid bacteria such as lactobacilli, despite the importance of this metal ion in the overall physiology of these bacteria (Bosma, Elleke F., et al. “Regulation and distinct physiological roles of manganese in bacteria.” FEMS Microbiology Reviews (2021)).

The present inventors found that the inactivation of MntR in lactobacilli increased the manganese scavenging activity yet without leading to cell death. Therefore, such bacteria can be advantageously exploited for its improved manganese scavenging ability. This is surprising, as it was known that excess accumulation of Mn²⁺ can easily lead to cytotoxicity primarily through mismetallation of proteins, as shown in the case of B. subtilis.

Useful lactobacilli may be provided by directly screening for wild type bacteria which lack functioning repression mechanism or by mutating relevant genes in wild type mother strain(s) and select therefrom mutants with higher manganese scavenging activity. One may for example mutate the mntR gene, its regulatory sequences or the binding site for the MntR, such as by substitution, truncation, deletion, point mutation, and/or knock-out.

### Manganese Scavenging Lactobacilli

The Lactobacillus strain(s) according to the present invention express the manganese transporter divalent metal cation transporter MntH1, which belongs to the family of TC #2.A.55.2.6. MntH1 is a manganese transporter known in the art that was identified to be important for manganese scavenging activity (Siedler et al. 2020).

The present application additionally provides exemplary MntH1 sequences listed as SEQ ID NO: 1-15. Preferably, the Lactobacillus strain(s) express the MntH1 transporter as set forth in SEQ ID NO: 1-15 or homologous sequences thereof.

MntH1 is found to be highly expressed in L. paracasei, L. rhamnosus, but in other Lactobacillus species is also possible. It is within a skilled person in the art to determine whether a given Lactobacillus expresses MntH1 transporter. For example, this can be determined using known methods, for example as described in the publication Siedler et al. 2020.

Preferably, the Lactobacillus strains belong to the species of L. salivarius, L. reuteri, L. brevis, L. kefiri, L. alimentarius, L. zeae, L. kimchicus, L. curvatus, L. sakei, L. casei, L. paracasei, L. rhamnosus, L. plantarum and L. fermentum. Most preferably, the Lactobacillus strains belong to the species of L. curvatus, L. sakei, L. casei, L. paracasei, L. rhamnosus, L. plantarum and L. fermentum.

In a preferred embodiment, the Lactobacillus strain comprises a polypeptide having at least 60%, at least 65%, at least 70%, at least 75%, at least 80%, at least 85%, at least 90%, at least 91%, at least 92%, at least 93%, at least 94%, at least 95%, 96%, at least 97%, at least 98%, at least 98%, or 100% sequence identity with the sequence of any one of SEQ ID NO: 1-15, preferably with SEQ ID NO: 1 or 2.

For purposes of the present invention, the degree of “sequence identity” between two polypeptide sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al. 2000, Trends Genet. 16: 276-277). One may use the EMBOSS Needle alignment as described in Madeira, Fábio, et al., “The EMBL-EBI search and sequence analysis tools APIs in 2019.” Nucleic acids research 47.W1 (2019): W636-W641. The optional parameters used are gap open penalty of 10, gap extension penalty of 0.5, and the EBLOSUM62 (EMBOSS version of BLOSUM62) substitution matrix. The output of Needle labeled “longest identity” (obtained using the nobrief option) is used as the percent identity and is calculated as follows:

(Identical Residues×100)/(Length of Alignment−Total Number of Gaps in Alignment)

Table 1 shows exemplary sequences which encode MntH1 and their sequence identity with SEQ ID NO: 1.

TABLE 1
MntH1 sequences

| | | | | % |
|---|---|---|---|---|
| | | | | identity |
| | | | | with |
| | SEQ | | | SEQ ID |
| Origin | ID NO | Protein ID | MntH1 Sequence | NO: 1 |
| Lactobacillus | 1 | WP_019888494.1 | MSDDHKKRHPIKLIQYANGPSLEEINGTVEVPH | — |
| paracasei | | | GKGFWRTLFAYSGPGALVAVGYMDPGNWSTSI | |
| NC_014334 | | | TGGQNFQYLLISVILMSSLIAMLLQYMAAKLGIV | |
| (L. paracasei | | | SQMDLAQAIRARTSKKLGIVLWILTELAIMATDI | |
| Zhang) | | | AEVIGAAIALYLLFHIPLVIAVLVTVLDVLVLLLLT | |
| | | | KIGFRKIEAIVVALILVILLVFVYQVALSDPNMGA | |
| | | | LLKGFIPTGETFASSPSINGMSPIQGALGIIGATV | |
| | | | MPHNLYLHSAISQTRKIDHKNPDDVAQAVKFSA | |
| | | | WDSNIQLSFAFVVNCLLLVMGVAVFKSGAVKDP | |
| | | | SFFGLFQALSDSSTLSNGVLIAVAKSGILSILFAV | |
| | | | ALLASGQNSTITGTLTGQVIMEGFVHMKMPLWA | |
| | | | RRLVTRIISVIPVIVCVMLTARDTPIQQHEALNTL | |
| | | | MNNSQVFLAFALPFSMLPLLMFTNSKVEMGDRF | |
| | | | KNTGWVKVLGWISVLGLTGLNLKGLPDSIAGFF | |
| | | | GDHPTATQTNMANIIAIVLIVAILALLAWTIWDL | |
| | | | YKGNQRYEAHLAAVADEKEAKADVDEQ | |
| Lactobacillus | 2 | | MSDDHKKRHPIKLIQYANGPSLEEINGTVEVPH | 99.8 |
| paracasei | | | GKGFWRTLFAYSGPGALVAVGYMDPGNWSTSI | |
| CHCC14676 | | | TGGQNFQYLLISVILMSSLIAMLLQYMAAKLGIV | |
| | | | SQMDLAQAIRARTSKKLGIVLWILTELAIMATDI | |
| | | | AEVIGAAIALYLLFHIPLVIAVLVTVLDVLVLLLLT | |
| | | | KIGFRKIEAIVVALILVILLVFVYQVALSDPNMGA | |
| | | | LLKGFIPTGETFASSPSINGMSPIQGALGIIGATV | |
| | | | MPHNLYLHSAISQTRKIDYKNPDDVAQAVKFSA | |
| | | | WDSNIQLSFAFVVNCLLLVMGVAVFKSGAVKDP | |
| | | | SFFGLFQALSDSSTLSNGVLIAVAKSGILSILFAV | |
| | | | ALLASGQNSTITGTLTGQVIMEGFVHMKMPLWA | |
| | | | RRLVTRIISVIPVIVCVMLTARDTPIQQHEALNTL | |
| | | | MNNSQVFLAFALPFSMLPLLMFTNSKVEMGDRF | |
| | | | KNTGWVKVLGWISVLGLTGLNLKGLPDSIAGFF | |
| | | | GDHPTATQTNMANIIAIVLIVAILALLAWTIWDL | |
| | | | YKGNQRYEAHLAAVADEKEAKADVDEQ | |
| Lactobacillus | 3 | WP_025013716.1 | MSDDNKKKHSMKLIQYANGPSLEEINGTVEVPH | 93.8 |
| casei | | | GKGFWRTLFAYSGPGALVAVGYMDPGNWSTSI | |
| | | | TGGQNFQYLLISVILMSSLIAMLLQYMAAKLGIV | |
| | | | SQMDLAQAIRARTSKTLGIVLWILTELAIMATDI | |
| | | | AEVIGAAIALYLLFHIPLVISVLITVLDVLVLLLLTK | |
| | | | IGFRKIEAIVVALILVILFVFIYQVALSDPNMGALL | |
| | | | KGFIPTSKTFANSPSVNGMSPIQGALGIIGATVM | |
| | | | PHNLYLHSAISQTRKIDHHDPDDVAQAVKFSA | |
| | | | WDSNIQLSFAFVVNCLLLVMGVAVFKSGAVKDP | |
| | | | SFFGLFEALSDSSTLSNGVLIAVAKSGILSILFAV | |
| | | | ALLASGQNSTITGTLTGQVIMEGFIHMKMPLWA | |
| | | | RRLVTRIISVIPVIVCVMLTARETPIQQHEALNTL | |
| | | | MNNSQVFLAFALPFSMLPLLMFTNSKVEMGDRF | |
| | | | KNTGWVKVLGWISVLGLTYLNLKGLPDSIAGFF | |
| | | | GDHPTAAQTAIANDIAYALIVAVLALLAWTVWD | |
| | | | LYKGNKRYEAHLEAVADAKEAKASNDVQ | |
| Lactobacillus | 4 | WP_099981497.1 | MKLIQYANGPSLEEINGTVEVPHGKGFWRTLFA | 93.6 |
| rhamnosus | | | YSGPGALVAVGYMDPGNWSTSITGGQNFQYLLI | |
| | | | SVILMSSLIAMLLQYMAAKLGIVSQMDLAQAIRA | |
| | | | RTSKKLGIVLWILTELAIMATDIAEVIGAAIALYLL | |
| | | | FHIPLVIAVLVTVLDVLVLLLLTKIGFRKIEAIVVA | |
| | | | LILVILLVFVYQVALSDPNMGALLKGFIPTGETFA | |
| | | | SSPSVNGMSPIQGALGIIGATVMPHNLYLHSAIS | |
| | | | QTRKIDHKDPEDVAQAVKFSAWDSNIQLTFAFV | |
| | | | VNCLLLVMGVAVFKSGAVKDPSFFGLFQALSDS | |
| | | | STLSNGVLIAVAKSGILSILFAVALLASGQNSTIT | |
| | | | GTLTGQVIMEGFIHMKMPLWARRLVTRVISVIPV | |
| | | | IVCVMLTARETPIQQHEALNTLMNNSQVFLAFAL | |
| | | | PFSMLPLLMFTNSKVEMGDRFKNTGWVKVLGW | |
| | | | VSVIGLTYLNLKGLPDSIAGFFGDNPTAAQTNIA | |
| | | | NMIAYVLIAAVLALLAWTIWDLYKGNKRYEAHLE | |
| | | | AVADEEEAKANDDVQ | |
| Lactobacillus | 5 | WP_112297335.1 | MSEKMNTPNRKHKLIEYANGPSLEEINGTIEVPK | 76.5 |
| plantarum | | | NLNFWKTLFAYSGPGALVAVGYMDPGNWSTSI | |
| | | | TGGQNYQYMLMSVILISSLIAMLLQYMAAKLGIV | |
| | | | SQMDLAQAIRARTSKSLGIVLWILTELAIMATDI | |
| | | | AEVIGAAIALYLLFNIPLVIAVFITVLDVLVLLLLTK | |
| | | | IGFRKIEAIVVCLILVILFVFVYQVALSNPDWGGV | |
| | | | IKGLVPTADTFSTSRSVNGMTPLSGALGIIGATV | |
| | | | MPHNLYLHSAISQTRKIDHNDEEDVARTVKFAA | |
| | | | WDSNIQLSFAFVVNSLLLIMGVAVFKSGAVKDP | |
| | | | SFFGLYEALSNTSMLSNGILISVAKSGALSALFAI | |
| | | | ALLASGQNSTITGTLTGQVIMEGFVHMRMPLWL | |
| | | | RRLVTRLISVIPVLICVLLTSGKSAIDEHTALNNL | |
| | | | MNNSQVFLAFALPFSMLPLLMMTDSAAEMGKRF | |
| | | | KNSLWIKGLGWLSVIGLTFLNLLGLPDSILGFFG | |
| | | | DNPSAGEQTFSKILAYLLIAAILALLVWTVFDLQR | |
| | | | GNKRYVEQQLAAAAKEANK | |
| Lactobacillus | 6 | WP_003700265.1 | MVNNENNHKKHKMIQYANGKSLEEVNGTVEIP | 72.7 |
| salivarius | | | KGKGFWKTLFAYSGPGALVAVGYMDPGNWSTS | |
| | | | ITGGQNFQYLLMSVILLSSLIAMLLQYMAAKLGI | |
| | | | VSQMDLAQAIRARTSKALGIVLWILTELAIMATD | |
| | | | IAEVIGAAIALYLLFDIPLIIAVFITVFDVLLLLLLTK | |
| | | | VGFRKIEAIVVCLIFVILFVFVYQVALSNPDWGG | |
| | | | VFKGLIPTSETFAKHPVVHDMSPLNGALGIIGAT | |
| | | | VMPHNLYLHSAISQTRKFDRNNEDDIANAVRFT | |
| | | | AWDSNIQLGLAFVVNSLLLIMGVAVFKSGAVED | |
| | | | PSFFGLYQALSDTSVMSNGLLAAAARTGILSTLF | |
| | | | AVALLASGQNSTITGTLTGQVIMEGFIHLRMPL | |
| | | | WARRLITRLLSVIPVLICVALTSGKSTIEEHEALN | |
| | | | NLMNNSQVFLAFALPFSMLPLVIMTGSKVEMGE | |
| | | | RFKNRLWINILGWISVISLTYLNMIGLPQNLEPFF | |
| | | | PADKVGLAHTVAYILIVLIIALLIWTLVELHLGNK | |
| | | | RFAAEQAKKHNK | |
| Lactobacillus | 7 | WP_003682262.1 | MRGGFGVDNTKNQHRKLRLIEHANGKSLEEIN | 68.6 |
| fermentum | | | GTVEVPHGKGFFRTLFAYSGPGALVAVGYMDPG | |
| | | | NWSTSITGGQSFQYTLMTTILISSLIAMLLQYMA | |
| | | | AKLGIVSQMDLAQAIRARTGKALGVILWLMTEL | |
| | | | AIMATDIAEVIGAAIALNLLFHIPLVLAVFITVLDV | |
| | | | LVLLLLTKIGFRKIEAIVACLILVILAVFAYQVALS | |
| | | | HPDWAGVFKGLLPTKEAIAKEPVVGGISPLTGSL | |
| | | | GIIGATVMPHNLYLHSAISQTRKIDHTNAEDIKQ | |
| | | | TVRFTAWDSNIQLTLAFFVNALLLIMGVAVFKNG | |
| | | | AVQDSSFFGLYDALNNTDMLSNGLLIAVAKSGV | |
| | | | LSTLFAIALLASGQNSTITGTLTGQVIMEGFVHM | |
| | | | KMPLWARRLITRLLSVVPVLVCVAMTAHESTID | |
| | | | QHASLNILMENSQVFLAFALPFSMLPLLIMTNSD | |
| | | | TEMGQFKNSLWVRVLGWISVIGLTFLNLYNLPQ | |
| | | | TYEGFGIWSKGLSDVLAWISIVVIVVLLAWTCFE | |
| | | | LIRGDRRLAAEREKHTWEK | |
| Lactobacillus | 8 | WP_016264550.1 | MNKVKGPKKHKLIEYANGPSLEEINGTVEVPEG | 74.6 |
| sakei | | | KTFWKTLLAYSGPGALVAVGYMDPGNWSTSIT | |
| | | | GGQSFQYLLMSVILVSSLIAMLLQYMAAKLGIVT | |
| | | | QMDLAQAIRARTSKSLGIVLWILTELAIMATDIA | |
| | | | EVIGAAIALYLLFNIPLVIAVFITILDVMLLLLLTKV | |
| | | | GFRKIEAIVVALIVVIFVVFAYEVALSNPDWAGVI | |
| | | | VGLVPTAKTFATTPNVGGMSPLTGALGIIGATVM | |
| | | | PHNLYLHSAISQTRKIDRNNEEQVAQTVRFSTW | |
| | | | DSNIQLTMAFFVNALLLIMGVAVFKTGAVKDPSF | |
| | | | FGLFEALSDTSTMSNGILASVARTGILSTLFAVA | |
| | | | LLASGQNSTITGTLTGQVIMEGFVHLRMPLWLR | |
| | | | RLVTRLLSVIPVLICVMMTSNKPPLEEHQALNTL | |
| | | | MNNSQVFLAFALPFSMLPLLMFTDSRVEMGDRF | |
| | | | KNSLVIRVLGWLSVIGLTYLNMLGLPGQIEAFFG | |
| | | | DHATAAQLALADHIAYVLIAAVLALLVWMIVELY | |
| | | | KGNQRFEQQLAAQAAE | |
| Lactobacillus | 9 | WP_003669360.1 | MISNLINKTQTKGGFGVDDTKNQHRKHKLIEYA | 70.1 |
| reuteri | | | NGKSLEEINGTVEVPRGKGFWRTLFAYSGPGAL | |
| | | | VAVGYMDPGNWSTSITGGQSFQYTLMTTILISS | |
| | | | LIAMLLQYMAAKLGIVSQMDLAQATRARTGKAL | |
| | | | GIILWIMTELAIMATDIAEVIGAAIALNLLFHIPLI | |
| | | | PSVFITVLDVLVLLLLTKIGFRKIEAIVACLILVILF | |
| | | | VFAYQVALSNPNWGGVFMGLLPSAKAIAQHPEI | |
| | | | GGITPLTGTLGIIGATVMPHNLYLHSAISQTRKID | |
| | | | HNDLDSIRQTVRFTTWDSNIQLSLAFIVNSLLLI | |
| | | | MGVAVFKTGAVQDSSFFGLYDALNNTSMLSNP | |
| | | | VLIAVAKSGVLSTLFAVALLASGQNSTITGTLTG | |
| | | | QVIMEGFIHMRMPLWARRLVTRIISVIPVIACVA | |
| | | | MTSGENTIQQHTALNLLMENSQVFLAFALPFSM | |
| | | | LPLLMMTNSEVEMGEFKNRGWVKVCGWISVIA | |
| | | | LTFLNLYNLPATYEGFGIWSKGTSDVLAYITIIVIL | |
| | | | ALLIWTCVELYKGDKRFAAEGKGFGQREAQMK | |
| | | | DSVVED | |
| Lactobacillus | 10 | WP_096109580.1 | MDLRKGVLKLSDNVQKKHKLISYANGRSLEEIN | 73.3 |
| brevis | | | GSVAVPKNISFWKALFMYSGPGALVAVGYMDP | |
| | | | GNWSTSITGGQNFQYLLMSIILISSLIAMLLQYM | |
| | | | AAKLGIVSQMDLAQAIRARTSKSLGIVLWIMTEF | |
| | | | AIMATDIAEVIGAAIALYLLFHIPLVIAVFITVFDV | |
| | | | LLLLLLTKIGFRKIEAIVVCLILVILVVFAYQVALS | |
| | | | NPDWGGVFAGLIPSPKTIASTPQIGGQTPITGAL | |
| | | | GIIGATVMPHNLYLHSAISQTRQINHDDEEDVA | |
| | | | RTVRFSSWDSNIQLTLAFFVNALLLIMGVAVFKS | |
| | | | GAVKDPSFFGLFQALSDTNTMSNGVLAGVAKT | |
| | | | GALSTLFAVALLASGQNSTITGTLTGQVIMEGFV | |
| | | | HMRMPLWLRRLVTRLISVIPVLICVMMTSGKSAI | |
| | | | DEHTALNDLMNNSQVFLAFALPFSMLPLLMMTD | |
| | | | SKLEMGERFKNSAWVKWLGWLSVLTLTGLNLY | |
| | | | NMPASIQGFYGDGITASETMTADVIAWVLNAAI | |
| | | | IALLVWTIYELRKGNRRLAQAVAADGKTN | |
| Lactobacillus | 11 | WP_056981840.1 | MPKEKQPKKQHLIHYANGPSLEEINGTIEVPKGR | 76.2 |
| kefiri | | | GFWKTLFMYSGPGALVAVGYMDPGNWSTSITG | |
| | | | GQNFEYLLMSVILLSSLIAMLLQYMAAKLGIVSQ | |
| | | | MDLAQAIRARTSKTLGIVLWILTELAIMATDIAE | |
| | | | VIGAAIALYLLFHIPLVYAVFITVFDVLLLLLLTKV | |
| | | | GFRKIEAIVVCLILVILFVFIYEVALSEPDFGAMVK | |
| | | | GLIPTGQTFSSADHVNGDTPLTGALGIIGATVMP | |
| | | | HNLYLHSAISQTRKVDHNDQDDVARTVRFSTW | |
| | | | DSNIQLSFAWVINSLLLVMGVAVFKTGAVKDPS | |
| | | | FFGLFDALSNTSMLSNGILIAVAKSGILSILFAVA | |
| | | | LLASGQNSTITGTLTGQVIMEGFIHMKMPLWLR | |
| | | | RLVTRLISVIPVLICVAMTSNETPIKQHEALNTLM | |
| | | | NNSQVFLAFALPFSMLPLLMMTDNAKEMGERFK | |
| | | | NTLWVKVLGWVSVLALTFLNMKGLPDNITSFFG | |
| | | | AAPSASQVSLAHTIAYVIIVAIVLLLLWTVYDLYS | |
| | | | SRNKMPQRFETTAEHYDESKKDKE | |
| Lactobacillus | 12 | WP_057737524 | MAEKHKLIEYANGPSLQEINGTVDVPKGKGFFK | 73.8 |
| alimentarius | | | TLFAYSGPGALVAVGYMDPGNWSTSITGGQNF | |
| | | | QYLLMSVILMSSLIAMLLQYMAAKLGIVSKMDLA | |
| | | | QAIRARTSRSLGIVLWILTELAIMATDIAEVIGGA | |
| | | | IALYLLFNIPLVIAVFITVGDVLVLLLLTKIGFRKIE | |
| | | | AIVVCLILVILFVFVYQVALSNPDWGGVFAGLIPT | |
| | | | GKTFATGPKIGGQTPLNGALGIIGATVMPHNLYL | |
| | | | HSAISQTRKVDHADEASVAQNVRFSAWDSNIQ | |
| | | | LTAAFFVNALLLIMGVAVFKSGAVEDPSFFGLYK | |
| | | | ALSDTSTLSNGVLIAVAKSGILSTLFAVALLASG | |
| | | | QNSTITGTLTGQVIMEGFVHMRMPLWLRRLVTR | |
| | | | LISVIPVLICVMLTSGKSAIDEHEALNTLMNNSQ | |
| | | | VFLAFALPFSMLPLLLMTDSATEMGNKFKNAAWI | |
| | | | KIFGWLSVIALTFLNLYGLPDQIKAFYGDGITSA | |
| | | | QSLQANIIAYVLIAAVLALLVWTVFDMHKGNERL | |
| | | | KTVLAKEDVTSTYEHLAKISASVSSEEDFDKQAT | |
| | | | AERNSEQR | |
| Lactobacillus | 13 | WP_128486151.1 | MNQQEKGKKHKLIEYANGPSLEEINGTVEVPEG | 72.9 |
| curvatus | | | KGFWKMLLAYSGPGALVAVGYMDPGNWSTSIT | |
| | | | GGQSFQYLLMSVILVSSLIAMLLQYMAAKLGIVT | |
| | | | QMDLAQAIRARTSKPLGIVLWILTELAIMATDIA | |
| | | | EVIGAAIALYLLFKIPLLIAVFITILDVMLLLLLTKI | |
| | | | GFRKIEAIVVALIVVIFVVFAYEVALSDPDWAGVI | |
| | | | VGLVPTAKTFATGPAVGGLTPLTGALGIIGATVM | |
| | | | PHNLYLHSAISQTRKIDRKNEAQVAQTVRFATW | |
| | | | DSNIQLTMAFFVNALLLIMGVAVFKTGTVKDPSF | |
| | | | FGLFKALSDTSTMSNGILASVARTGILSTLFAVA | |
| | | | LLASGQNSTITGTLTGQVIMEGFIHLRMPLWLRR | |
| | | | LVTRLLSVIPVLICVMMTSNKPALEEHEALNTLM | |
| | | | NNSQVFLAFALPFSMLPLLMFTDSRVDMGDRFK | |
| | | | NSWLIKSLGWLSVIGLTYLNMMGLPGQIEAFYG | |
| | | | DHASAAQLATADRIAYVLIAGVMALLVWMIIELY | |
| | | | KGNKRFEQQLATEN | |
| Lactobacillus | 14 | WP_070650615.1 | MSDDHKKKHSMKLIQYANGPSLEEINGTVEVPH | 94.2 |
| zeae | | | GKGFWRTLFAYSGPGALVAVGYMDPGNWSTSI | |
| | | | TGGQNFQYLLISVILMSSLIAMLLQYMAAKLGIV | |
| | | | SQMDLAQAIRARTSKTLGIVLWILTELAIMATDI | |
| | | | AEVIGAAIALYLLFHIPLVISVLITVLDVLVLLLLTK | |
| | | | IGFRKIEAIVVALILVILFVFIYQVALSDPNMGALL | |
| | | | KGFIPTSETFANSPSVNGMSPIQGALGIIGATVM | |
| | | | PHNLYLHSAISQTRKIDHHDPDDVAQAVKFSA | |
| | | | WDSNIQLSFAFVVNCLLLVMGVAVFKSGAVKDP | |
| | | | SFFGLFEALSDSSTLSNGVLIAVAKSGILSILFAV | |
| | | | ALLASGQNSTITGTLTGQVIMEGFIHMKMPLWA | |
| | | | RRLVTRIISVIPVIVCVMLTARETPIQQHEALNTL | |
| | | | MNNSQVFLAFALPFSMLPLLMFTNSKVEMGERF | |
| | | | KNTGWVKVLGWISVLGLTYLNLKGLPDSIAGFF | |
| | | | GDHPTATQTTIANDIAYALIVAVLALLAWTIWDL | |
| | | | YKGNKRYEAHMEAVADAKEAKASNDVQ | |
| Lactobacillus | 15 | WP_056942608.1 | MKDQNTPRKHHLIEYANGKSLEEINGTVEVPKG | 78.8 |
| kimchicus | | | RGFWRTLFMYSGPGALVAVGYMDPGNWSTSIT | |
| | | | GGQNFQYLLMSVILMSSLIAMLLQYMAAKLGIV | |
| | | | SQMDLAQAIRARTSKSLGVVLWILTELAIMATDI | |
| | | | AEVIGAAIALYLLFHIPLVYAVFITVFDVLLLLLLTK | |
| | | | VGFRKIEAIVVCLILVILFVFVYQVALSNPNWAAV | |
| | | | IGGLVPTGETFSSSPSVGGMTPISGALGIIGATV | |
| | | | MPHNLYLHSAVSQSRKIDHNDEEDVARTVRFST | |
| | | | WDSNIQLSFAFVVNSLLLIMGVAVFKTGAVKDP | |
| | | | SFFGLFEALSNTSTLSNGVLIGVAKSGVLSVLFA | |
| | | | VALLASGQNSTITGTLTGQVIMEGFVHMRMPLW | |
| | | | LRRLVTRLISVIPVLICVAITGRETPIQQHEALNN | |
| | | | LMNNSQVFLAFALPFSMLPLLMMTNSRLEMGQR | |
| | | | FKNNFLVKLFGWISVIALTFLNMKGLPGSIAGFY | |
| | | | GDNITAAQTHQANIIAYILIAAVLALLVWTVYDLY | |
| | | | KGNQRLAAKLAAEPSNNDVAD | |

### Inactivated MntR and MntR Binding Site

The Lactobacillus strains of the present application are characterized by inactivated MntR or inactivated binding site for MntR located upstream of the mntH1 gene, which means the lack of repression of the mntH1 transcription. Inactivation of MntR or its binding site can be carried out using methods known to a skilled person in the art, for example, by substitution, truncation, deletion, point mutation and/or knock-out.

MntR, when activated by Mn²⁺, acts as repressor and binds to an operator site (also referred to as the “binding site for MntR” or simply the “binding site”) in the vicinity of the promoter region for mntH1 and thereby represses the transcription of mntH. The binding site may be located between the promoter elements and start codon. The binding site is highly conserved in lactobacilli and has a sequence motif listed as SEQ ID NO: 16 with the polynucleotide sequence of DDDKWWRSKNNNCHWAMMA (where M represents A or C; R represents A or G; W represents A or T; S represents C or G; K represents G or T; H represents A, C or T; D represents A, G or T; N represents A, C, G or T). The sequence motif was prepared based on TF binding site data among multiple bacterial species identified SEQ ID NO: 17-30 (RegPresice, Novichkov et al. “RegPrecise 3.0-a resource for genome-scale exploration of transcriptional regulation in bacteria.” BMC genomics 14.1 (2013): 1-12) shown in Table 2.

TABLE 2
MntR binding site sequences and sequence motif

| Sequence origin | SEQ ID NO | Binding site sequences |
|---|---|---|
| Sequence Motif | 16 | DDDKWWRSKNNNCHWAMMA |
| Lactobacillus brevis ATCC 367 | 17 | ATTTTTGGTAAGCCAAAAAT |
| Lactobacillus brevis ATCC 367 | 18 | AAGGAAGGGAGTCTTAAAAT |
| Lactobacillus casei ATCC 334 | 19 | GAATTAGGTCACCCTAAAAA |
| Lactobacillus casei ATCC 334 | 20 | AAGTTAGGGAGACCTAAAAG |
| Lactobacillus fermentum IFO 3956 | 21 | TTTTTATGCTAACCTAACAA |
| Lactobacillus plantarum WCFS1 | 22 | AAGTTAACTGCACCTAACAA |
| Lactobacillus plantarum WCFS1 | 23 | TTTGTAGGCATACCTAAAAA |
| Lactobacillus reuteri JCM 1112 | 24 | TTTTTATGTTACCCTAACAA |
| Lactobacillus rhamnosus GG | 25 | AAGTTAGGGAGACCTAAAAG |
| Lactobacillus rhamnosus GG | 26 | GAATTAGGTCACCCTAAAAT |
| Lactobacillus sakei subsp. sakei 23K | 27 | AAGTTAGGTATACCTAAAAG |
| Lactobacillus sakei subsp. sakei 23K | 28 | AAGTTAGGGCATCCTAAAAT |
| Lactobacillus sakei subsp. sakei 23K | 29 | AAGTTAAGGGACCCAAAAAG |
| Lactobacillus salivarius subsp. | 30 | AGTTAAGGTAGACCTAAAAA |
| salivarius UCC118 | | |

The sequence motif can be described as the position-specific probability matrix shown in Table 3.

TABLE 3
position-specific probability matrix for the sequence motif

| Position | A | C | G | T |
|---|---|---|---|---|
| 1 | 0.642857 | 0.000000 | 0.142857 | 0.214286 |
| 2 | 0.642857 | 0.000000 | 0.071429 | 0.285714 |
| 3 | 0.142857 | 0.000000 | 0.642857 | 0.214286 |
| 4 | 0.000000 | 0.000000 | 0.071429 | 0.928571 |
| 5 | 0.142857 | 0.000000 | 0.000000 | 0.857143 |
| 6 | 0.928571 | 0.000000 | 0.000000 | 0.071429 |
| 7 | 0.142857 | 0.000000 | 0.857143 | 0.000000 |
| 8 | 0.000000 | 0.071429 | 0.928571 | 0.000000 |
| 9 | 0.000000 | 0.000000 | 0.428571 | 0.571429 |
| 10 | 0.500000 | 0.214286 | 0.142857 | 0.142857 |
| 11 | 0.500000 | 0.071429 | 0.285714 | 0.142857 |
| 12 | 0.428571 | 0.214286 | 0.214286 | 0.142857 |
| 13 | 0.000000 | 1.000000 | 0.000000 | 0.000000 |
| 14 | 0.142857 | 0.785714 | 0.000000 | 0.071429 |
| 15 | 0.142857 | 0.000000 | 0.000000 | 0.857143 |
| 16 | 1.000000 | 0.000000 | 0.000000 | 0.000000 |
| 17 | 0.928571 | 0.071429 | 0.000000 | 0.000000 |
| 18 | 0.928571 | 0.071429 | 0.000000 | 0.000000 |
| 19 | 1.000000 | 0.000000 | 0.000000 | 0.000000 |

Inactivation of the binding site can be carried out using methods known to a skilled person in the art to render it non-functional. This is preferably carried out by truncation, full or partial deletion and/or knock-out.

As used herein, “inactivation” within the spirit of the present invention refers to the inability of MntR to bind to the operator site (“binding site”) which is located in the vicinity of the promoter region for mntH1 in the presence of sufficient manganese. This may be for example due to lack of functional MntR or functional binding site for MntR. “Inactivation” can be determined according to methods known in the art, such as the electrophoretic mobility shift assay (EMSA) which can be used for studying DNA-protein interactions. This technique is based on the fact that DNA-protein complexes migrate slower than non-bound DNA in a native polyacrylamide or agarose gel, resulting in a “shift” in migration of the labeled DNA band. For this, the Thermo Scientific LightShift Chemiluminescent EMSA Kit can be used following the manufacturers protocol. To determine whether a given MntR is able to bind to an operator site or not, the test can be carried out with amplified DNA containing the operator site and a solution containing the MntR protein, in different manganese concentrations ranging from 0 mg/L to 20 mg/L. As a reference control condition, the DNA sequence containing SEQ ID NO: 20 and the MntR with the SEQ ID NO: 31 should be used. Reduction in the ability to bind at a manganese concentration of 0.135 mg/L or higher compared to the reference condition is considered inactivation.

Another way to determine the inactivity of the MntR protein is to analyze the mntR gene sequence to see if it comprises a modification that may cause inactivation of the protein, for example, based on folding predictions.

MntR is a homologue of DtxR that is a well-characterized, divalent metal ion-dependent repressor that controls iron transport functions in C. diphtheriae. Structurally, MntR forms binuclear complexes with Mn²⁺ at two binding sites, labeled A and C, that are separated by 4.4 Å. (Kliegman, Joseph I., et al. “Structural basis for the metal-selective activation of the manganese transport regulator of Bacillus subtilis.” Biochemistry 45.11 (2006): 3493-3505; McGuire et al. “Roles of the A and C sites in the manganese-specific activation of MntR.” Biochemistry 52.4 (2013): 701-713). The structure of MntR and related proteins have been studied by Chen et al., 2017 (“Molecular insights into hydrogen peroxide-sensing mechanism of the metalloregulator MntR in controlling bacterial resistance to oxidative stresses.” Journal of Biological Chemistry 292.13 (2017): 5519-5531).

A mutation may be the occurrence of a premature stop codon, or an insertion that e.g. cause frame shift, a deletion, a mutation etc. In preferred embodiment, the mutation occurs on the cysteine residues that are present in the MntR on the N-terminal DNA binding domain, the C-terminal dimerization domain or the metal binding site located in between.

It should also be understood that if a given strain does not express MntR, such as due to the lack of the mntR gene, it comprises inactivated MntR. This is the case for the mutant as exemplified in the present application.

### Improvement of Manganese Scavenging Activity

Based on the finding, the inventors provide a strategy to improve manganese scavenging activity of a Lactobacillus strain. As defined herein, the term “manganese scavenging activity” or “manganese uptake activity” refers to the ability to import free manganese by bacteria when cultured in a condition which allows for that. “Improved manganese scavenging activity” can be observed through the ability to take up manganese at a manganese concentration of 0.135 mg/L or higher. This can be determined as follows: The strain to be analyzed are grown in pasteurized cow milk for 24 hours at 37° C. (cow milk would contain intrinsic manganese, which is generally around 0.06 mg/L but may vary depending on the milk). Afterwards, two replicates of the fermented milk (150 μl) are transferred to a 96 microtiter plate and to half of the samples manganese is added to a final concentration of 6 mg/L and to the other half a final concentration of 0.135 mg/L (taking into account of manganese already present in the milk, which should be determined). Afterwards, 50-100 CFU of D. hansenii (e.g. CHCC16374) per gram product are inoculated to the fermented milk with and without manganese, to determine if manganese is depleted. After 4 days of incubation at 17° C., a dilution row of the samples is spotted on selective YGC agar plates to analyze the yeast growth. The yeast growth can be enumerated by optical inspection. If differences between with 0.135 mg/L and 6 mg/L are observed, improved manganese scavenging is shown.

Where mutation is carried out, the increase may be achieved by obtaining mutants in which the MntR protein or corresponding binding site is inactivated and selecting from the mutant daughter strains whose manganese scavenging activity is increased compared to the mother strain. In preferred embodiments, the daughter Lactobacillus strains has higher manganese scavenging activity compared to the mother strain in milk having a manganese concentration of 0.135 mg/L, 0.2 mg/L, 0.5 mg/L or 1.0 mg/L.

The methods of the present invention comprise the following steps:
providing one or more Lactobacillus strains which express the manganese transporter MntH1 as the mother strain,
obtaining one or more mutants from the mother strain, in which the manganese transporter regulator MntR or its binding site upstream of mntH1 is inactivated, and
selecting from the obtained mutants one or more daughter Lactobacillus strains having higher manganese scavenging activity is increased compared to the mother strain.

The term “expresses the MntH1 protein” refers to the ability to express said protein when the cell is in a viable state.

In one preferred embodiment, the method comprises the following steps:
providing one or more Lactobacillus strains which express the manganese transporter MntH1 as the mother strain,
mutating gene(s) in the mother strain which encode the MntR or regulate the expression of genes which encode the MntR, or mutating binding site for MntR upstream of mntH1, preferably by substitution, truncation, deletion, point mutation and/or knock-out to obtain one or more mutants from the mother strain, in which the manganese transporter regulator MntR or its binding site upstream of mntH1 is inactivated, and
selecting from the obtained mutants one or more daughter Lactobacillus strains having higher manganese scavenging activity compared to the mother strain,

The comparison is made for the mother and daughter strain under the same condition, preferably in a suitable medium with a predetermined manganese concentration, such as a manganese concentration of 0.135 mg/L or higher. The manganese concentration may be predetermined depending on the manganese scavenging ability of the mother strain, as well as the type of food product and the amount of manganese to be scavenged in the food product which the daughter strain is intended for.

In another embodiment, the present method comprises:
providing one or more Lactobacillus strains which express the manganese transporter MntH1 as the mother strain,
mutating in the mother strain gene(s) which encode the MntR protein and/or which regulate the expression of genes which encode the MntR protein by deleting fully or partially said gene(s),
obtaining one or more mutants from the mother strain, in which the MntR or its binding site upstream of mntH1 is inactivated, and
selecting from the obtained mutants one or more daughter Lactobacillus strains with higher manganese scavenging activity compared to the mother strain.

The manganese scavenging activity may be evaluated by its ability to inhibit the yeast Debaryomyces hansenii. In preferred embodiments, the selected daughter strain exhibits higher inhibitory activity towards Debaryomyces hansenii than the mother strain, compared under the same condition, preferably in manganese concentration of 0.135 mg/L or higher.

A further aspect of the invention provides composition(s) comprising one or more daughter strains obtained by the present method as disclosed herein.

### MntR Sequences

A suitable mother stain according to the present invention comprises the manganese transport regulator MntR which is a transcription factor for mntH1 gene. It should be understood that the MntR protein of the mother strain is “functionally active.” MntR has been studied in detail at the molecular level, for example by Chen et al 2017.

Table 4 shows exemplary sequences which encode MntR and their sequence identity with SEQ ID NO: 31.

TABLE 4
MntR sequences

| | | | | % |
|---|---|---|---|---|
| | | | | identity |
| | SEQ | | | with SEQ |
| Origin | ID NO | Protein ID | MntR Sequence | ID NO: 31 |
| Lactobacillus | 31 | WP_019885993.1 | MTPNKEDYLKLIFEIGGDTQLVSNKQIVA | — |
| paracasei | | | GMHVSAASVSEMINKLGEEKLVAHTPYQ | |
| | | | GIQLTSAGRKKAAILVRNHRLWEVFLVQ | |
| | | | CLKYPADAVHQEAEKLEHALTPEMAKRL | |
| | | | SAMLGEPRYCPHGGVIPDANGHYLQQSR | |
| | | | VTLGTLDVGQSGHIERVIDEVSLIDYTVK | |
| | | | IDLRLDDEFTVTAKTLDAVIIKLARTGKEL | |
| | | | AVDADRADHIFVEL | |
| Lactobacillus | 32 | WP_025012314.1 | MTPNKEDYLKLIFEIGGDTELVSNKQIVA | 93.5 |
| casei | | | GMHVSAASVSEMINKLGEEKLVAHTPYQ | |
| | | | GIQLTSAGRKKAAILVRNHRLWEVFLVK | |
| | | | CLKYSPDAVHQEAEKLEHALTPEMAKRL | |
| | | | SAMLGNPEYCPHGGVIPDAEGHYIQQSR | |
| | | | VTLGTMEVGQRGHIERVIDEVSLIDYTVK | |
| | | | IDLRLDDAFTVTAKTLDAVIIKLDRTGKEL | |
| | | | AVDADRAAHIFVEL | |
| Lactobacillus | 33 | WP_005685509.1 | MTPNKEDYLKLIFEIGGDTELVSNKQIVA | 92.1 |
| rhamnosus | | | GMHVSAASVSEMINKLGEEKLVAHTPYQ | |
| | | | GIQLTSAGRKKAAILVRNHRLWEVFLVK | |
| | | | CLKYPADAVHQEAEKLEHALTPEMAKRL | |
| | | | AAMLGNPQYCPHGGVIPDADGHYIQQS | |
| | | | RVTLGAMEVGQKGHIERLIDEVSLIDYTV | |
| | | | KLDLRLDDVFTVTAKTLDAVVIKLDRTGK | |
| | | | ELAVDADRAAHIFVEL | |
| Lactobacillus | 34 | WP_003641226.1 | MTPMKEDYLKIIFELGGTKKKVSNKQIAL | 44.9 |
| plantarum | | | SLDIAAGSVTEMVGKLVQEGLAKHTPYA | |
| | | | GISLTKKGIRYAETLVRKHRIWEDFLVDK | |
| | | | LDYDLPDVHTEAEVLEHVTSERLVDSLEA | |
| | | | FLGNPTHCPHGGAIPDKDGHYQEDSHV | |
| | | | SLADTQDGTTVTIERFIDNHDLLVYLHDT | |
| | | | PLKIGQQVTVLKHDPFEGPVTVSIQKTGE | |
| | | | EIPVSYKAAHNVFVK | |
| Lactobacillus | 35 | WP_003701679.1 | MTPKKEDYLKIIFELGGTKKKVSNKQIAM | 46.8 |
| salivarius | | | SLNVAAGSVTEMVNKLVKEGLAAHTPYA | |
| | | | GISLTDEGIEVAEKLVRRHRLWETFLVEK | |
| | | | LDYQLSEVHDEAEVLEHVASDKLMKKLD | |
| | | | QFLNSPRECPHGGVIPTEAGEYEEESHEY | |
| | | | LAEIKVGETVEVDRFIDNHELLTYLDDLEL | |
| | | | KLGDKIEVLEHLPFEGPIKVKRLADGAEL | |
| | | | SIGYKAAHYIFVK | |
| Lactobacillus | 36 | WP_012391612.1 | MTPMKEDYLKIIFELGGGRKKVSNKEISL | 44.7 |
| fermentum | | | GLGIAAGSVTEMITKLADEGLVEHEPYAG | |
| | | | IALTEKGGRYAAELVRKHRLWETFLVDKL | |
| | | | HYNMTDVHPEAEILEHKTSDHLATALDD | |
| | | | FLGHPAYCPHGGVIPSANGRFTNISHRLL | |
| | | | AEGEDGEEVIIERFLDNHDLLTYLSEIGLR | |
| | | | LRDYIKIVKHEPFEGPVVVERLTDGQTLN | |
| | | | ISYKAAHNVFITPKDK | |
| Lactobacillus | 37 | WP_011374089.1 | MTPNKEDYLKIIFELGGDAKKVTNKEILA | 53.2 |
| sakei | | | GLNVSAASVTEMVNKLVKENYVNHTPYQ | |
| | | | GIQLTSEGAREAALLVRNHRLWEVFLVD | |
| | | | KLHYQFNTVHPEAEQLEHVTNHDLAERL | |
| | | | ADFLGHPKRCPHGGIIPNAKGEFEQQSH | |
| | | | HALVDLEVGEKAVIERVLDDNDLLKYTLE | |
| | | | IGLTVGDTVTLTKVGLFESPITVMDETQQ | |
| | | | TEIQVGIKAAQHIFVTPIAAD | |
| Lactobacillus | 38 | WP_003668809.1 | MTPMKEDYLKIIFELGGSHKKVSNKEISL | 42.9 |
| reuteri | | | GLGIAAGSVTEMISKLADEGLVVHEPYA | |
| | | | GISLTEKGQKYAAELVRKHRLWETFLVD | |
| | | | KLHYNFADVHSEAEILEHQTSDRLATALD | |
| | | | SFLQHPDHCPHGGVIPSANGKFPDVTHR | |
| | | | LLADADDGEKVELERFLDNHELLTYLEEL | |
| | | | GLRPQEQVTVIRHEPFEGPIVIQKENNDQ | |
| | | | EINVSYKASHNIFIEPDTAQENKD | |
| Lactobacillus | 39 | WP_035464306.1 | MTPMKEDYLKIIFELGGRQKKVSNKQIAI | 44.4 |
| brevis | | | SLNIAAGSVTEMVNKMAAEGLAEHTPYA | |
| | | | GISLTNRGIRLAEDLVRKHRIWEDFLVEK | |
| | | | LGYALPDVHDEAEVLEHVTSPKLIDALDD | |
| | | | MLGNPTHCPHGGVIPDRQGHYHEDSHT | |
| | | | VLNDAADGEIVTVDRFIDNHDLLTYLGDL | |
| | | | KLDIGDQLQVLKHDPFEGPVTVQNLTDN | |
| | | | AELIVSYKAAHYIFVK | |
| Lactobacillus | 40 | WP_054769781.1 | MTPMKEDYLKIIFELGGSDDLVSNKQIAI | 41.6 |
| kefiri | | | SLNIAAGSVTEMVNKLVEEKLVTHEPYSG | |
| | | | VQLTKKGKKYAEELVRKHRIWETFLANTL | |
| | | | HYDISDVHDEAELLEHVTSDKMIDHLDD | |
| | | | FLGNPKRCPHGGVIPDRNGNYHPDKDKL | |
| | | | LTDAKDGEEVVVNRFIDNHDLLTLLGDIK | |
| | | | LDIGDKLKIISHDPFEGSVTVKNLTDKKK | |
| | | | LVIGFKTAHYVFVR | |
| Lactobacillus | 41 | WP_057738330.1 | MSPNKENYLKTIYELNYDFTKITNKRISEI | 40.7 |
| alimentarius | | | MNVSAPSVTEMLNSLSSEGYLTHTPYNKI | |
| | | | VLTPKGNKVSEKLVRTHRLWEVFLHECL | |
| | | | KYPVDNVHHNADALEHASDDGLIDHLN | |
| | | | DFLDHPQRCPHGGIIPGNGQGETDADD | |
| | | | KLLSMIPDNTKVQIVRVSDNYDFLQYFG | |
| | | | SLNLEIDDTIEVLKHEKFDNSLVVKKEDG | |
| | | | TKLTIGAKAIDYIFVELR | |
| Lactobacillus | 42 | WP_035185906.1 | MTPNKEDYLKIIFELGGDAKKVTNKEILA | 52.3 |
| curvatus | | | GLNVSAASVTEMVNKLVKENYVNHTPYQ | |
| | | | GIQLTSEGAREAALLVRNHRLWEVFLVD | |
| | | | KLHYQFNTVHPEAEQLEHVTNHDLAERL | |
| | | | ADFLGHPTRCPHGGIIPNAKGEFEQQSHI | |
| | | | ALETLQVGETAIIDRVLDDNDLLKYTLEIG | |
| | | | LSVGDSVTLQKVGLFESPLTVFNNTSQTE | |
| | | | IQIGLKAAQHIFVTPQN | |
| Lactobacillus | 43 | WP_010493697.1 | MTPNKEDYLKLIFEIGGDTELVSNKQIVA | 92.1 |
| zeae | | | GMHVSAASVSEMINKLGEEKLVAHTPYQ | |
| | | | GIQLTSAGRKKAAILVRNHRLWEVFLVK | |
| | | | CLKYSPDAVHQEAEKLEHALTPEMAKRL | |
| | | | SAMLGNPEYCPHGGVIPDAEGHYIQQSR | |
| | | | VTLGAMEVGQRGHIERLIDEVSLIDYTVK | |
| | | | IDLRLDDAFTVTAKTLDAVVIKLDRTGKE | |
| | | | LAVDADRAAHIFVEL | |
| Lactobacillus | 44 | WP_056942173.1 | MTPMKEDYLKLIFEIGGGSQKVSNKQIAI | 45.0 |
| kimchicus | | | SLDIAAGSVTEMVTKMAAEGLVEHEPYA | |
| | | | GISLTETGAKLAVELVRKHRIWETFLVSE | |
| | | | LKYALPDIDDDAEKLEHVTSTKLLNALDD | |
| | | | LLGHPKRCPHGGVIPDRNGHYEEDSHRI | |
| | | | LNDVKDGETVVVDRFIDNRDLLNYLGDI | |
| | | | KLDLGDQLQVIKHDSFEGPILVENLTDD | |
| | | | SELSIGYKAAHYIFVK | |

Since the transcription factor MntR is pervasive among lactobacilli, it is generally expected that the transcription factor would be present and functionally active in the bacteria, i.e. acting as a repressor for the mntH1 gene. As a repressor, it would bind to the corresponding binding site upstream of the mntH1 gene to prevent transcription. The term “upstream” refers to a location which is towards the 5′ end of the polynucleotide from a specific reference point. A skilled person in the art understands that the binding site is operably linked to the mntH1 gene and the distances in between may vary depending on the bacterium. For example, the binding site and the start codon may be less than 500 base pairs apart, such as less than 400 base pairs apart, such as less than 300 base pairs apart.

For example, the MntR protein of the mother strain may have at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98% or 100% sequence identity with any one of the sequences of SEQ ID NO: 31-44.

Preferably, the MntR protein of the mother strain may have at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98% or 100% sequence identity with any one of the sequences of SEQ ID NO: 31.

Obtaining Mutants from the Mother Strain

From a lactobacillus which comprise the manganese transporter MntH1 as the mother strain, it is possible to obtain one or more mutants in which MntR is inactive. This may be due to the lack of functional MntR or functional binding site for MntR.

In the present context, the term “mutant” should be understood as a strain derived, or a strain which can be derived, from a strain of the invention or the mother strain by means of e.g. genetic engineering, radiation and/or chemical treatment. Mutants can be obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light, or to a spontaneously occurring mutant. A mutant may have been subjected to several mutagenization treatments (a single treatment should be understood one mutagenization step followed by a screening/selection step), but it is presently preferred that no more than 20, or no more than 10, or no more than 5, treatments (or screening/selection steps) are carried out. In a presently preferred mutant, less than 5%, or less than 1% or even less than 0.1% of the nucleotides in the bacterial genome have been exchanged with another nucleotide, or deleted, compared to the mother strain.

Mutation is preferably introduced on the cysteine residues that are present in the MntR. Mutation can also be made on the N-terminal DNA binding domain, the C-terminal dimerization domain or the metal binding site located in between.

Inactivation of MntR can be carried out by various means. The protein may be inactivated by suitable modification introduced into the mntR gene, including, but not limited to, an insertion that e.g. causes frame shift, a stop codon, deletion or substitution. It is within the scope of the present application that the mutation would also include mutation in the regulatory sequences which control the expression of the MntR. Such mutations will lead to a decrease or absence of MntR expression. For instance, introducing a stop codon or a frameshift insertion in the mntR gene could give a non-functional gene that would e.g. either express no MntR protein or express a partial length inactive MntR protein.

In particular, DNA recombinant technology could be used. Other routine methods to introduce mutation is by homologous recombination of a suitable DNA fragment into the gene sequence (e.g. by use of the publicly available pGhost vectors or by other cloning vectors). The introduced fragment may contain for instance a nonsense (stop) codon, a frameshift mutation, a deletion, a mutation or an insertion. In some embodiments, the mutation includes a N-terminal deletion or a C-terminal deletion. It is routine work for the skilled person to choose an adequate strategy to e.g. introduce a suitable modification of the mntR gene to inactivate the MntR protein. Alternatively, one may randomly mutagenize (e.g. by UV radiation) and select for mutations wherein the MntR protein or relevant sequences are inactivated. Both genetically modified techniques as well as non-genetically modified techniques may be used in the present application. Genetically modified techniques offer a straight-forward modification, whereas non-genetically modified strategies are preferred if regional rules or market demands require so.

Lactobacilli with Inactivated MntR

The present application further includes lactobacilli obtained or obtainable by the presently disclosed methods. It is also possible to provide such strains by selecting from wild type strains those with inactivated MntR.

In preferred embodiments, the MntR protein is inactivated, for example due to a frameshift or a stop codon sequence encoding the protein. Useful strains preferably belong to the species of L. salivarius, L. reuteri, L. brevis, L. kefiri, L. alimentarius, L. zeae, L. kimchicus, L. curvatus, L. sakei, L. casei, L. paracasei, L. rhamnosus, L. plantarum and L. fermentum. More preferably, the Lactobacillus strains belong to the species of L. curvatus, L. sakei, L. casei, L. paracasei, L. rhamnosus, L. plantarum and L. fermentum.

The present application thus provides lactobacillus strains belonging to the species of L. salivarius, L. reuteri, L. brevis, L. kefiri, L. alimentarius, L. zeae, L. kimchicus, L. curvatus, L. sakei, L. casei, L. paracasei, L. rhamnosus, L. plantarum and L. fermentum comprising the manganese transporter MntH1, inactivated manganese transporter regulator MntR and/or inactivated binding site for MntR upstream of mntH1. The MntH1 is preferably a polypeptide having at least 60%, at least 65%, at least 70%, at least 75%, at least 80%, at least 85%, at least 90%, at least 95%, at least 97%, at least 98% or 100% sequence identity with any one of the sequences of SEQ ID NO: 1-15.

In preferred embodiments, the present application provides a L. paracasei, comprising the manganese transporter MntH1, inactivated manganese transporter regulator MntR and/or inactivated binding site for MntR upstream of mntH1, wherein the MntH1 is preferably a polypeptide having at least 80%, at least 85%, at least 90%, at least 95%, at least 97%, at least 98% or 100% sequence identity with any one of the sequences of SEQ ID NO: 1 or 2.

In another preferred embodiments, the present application provides a lactobacillus strain, preferably L. casei, comprising the manganese transporter MntH1, inactivated manganese transporter regulator MntR and/or inactivated binding site for MntR upstream of mntH1, wherein the MntH1 is preferably a polypeptide having at least 80%, at least 85%, at least 90%, at least 95%, at least 97%, at least 98% or 100% sequence identity with any one of the sequences of SEQ ID NO: 3.

In another preferred embodiments, the present application provides a lactobacillus strain, preferably L. rhamnosus, comprising the manganese transporter MntH1, inactivated manganese transporter regulator MntR and/or inactivated binding site for MntR upstream of mntH1, wherein the MntH1 is preferably a polypeptide having at least 80%, at least 85%, at least 90%, at least 95%, at least 97%, at least 98% or 100% sequence identity with any one of the sequences of SEQ ID NO: 4.

In another preferred embodiments, the present application provides a lactobacillus strain, preferably a L. plantarum, comprising the manganese transporter MntH1, inactivated manganese transporter regulator MntR and/or inactivated binding site for MntR upstream of mntH1, wherein the MntH1 is preferably a polypeptide having at least 80%, at least 85%, at least 90%, at least 95%, at least 97%, at least 98% or 100% sequence identity with any one of the sequences of SEQ ID NO: 5.

In another preferred embodiments, the present application provides a lactobacillus strain, preferably L. salivarius, comprising the manganese transporter MntH1, inactivated manganese transporter regulator MntR and/or inactivated binding site for MntR upstream of mntH1, wherein the MntH1 is preferably a polypeptide having at least 80%, at least 85%, at least 90%, at least 95%, at least 97%, at least 98% or 100% sequence identity with any one of the sequences of SEQ ID NO: 6.

In another preferred embodiments, the present application provides a lactobacillus strain, preferably L. fermentum, comprising the manganese transporter MntH1, inactivated manganese transporter regulator MntR and/or inactivated binding site for MntR upstream of mntH1, wherein the MntH1 is preferably a polypeptide having at least 80%, at least 85%, at least 90%, at least 95%, at least 97%, at least 98% or 100% sequence identity with any one of the sequences of SEQ ID NO: 7.

In another preferred embodiments, the present application provides a lactobacillus strain, preferably L. sakei, comprising the manganese transporter MntH1, inactivated manganese transporter regulator MntR and/or inactivated binding site for MntR upstream of mntH1, wherein the MntH1 is preferably a polypeptide having at least 80%, at least 85%, at least 90%, at least 95%, at least 97%, at least 98% or 100% sequence identity with any one of the sequences of SEQ ID NO: 8.

In another preferred embodiments, the present application provides a lactobacillus strain, preferably L. reuteri, comprising the manganese transporter MntH1, inactivated manganese transporter regulator MntR and/or inactivated binding site for MntR upstream of mntH1, wherein the MntH1 is preferably a polypeptide having at least 80%, at least 85%, at least 90%, at least 95%, at least 97%, at least 98% or 100% sequence identity with any one of the sequences of SEQ ID NO: 9.

In another preferred embodiments, the present application provides a lactobacillus strain, preferably L. brevis, comprising the manganese transporter MntH1, inactivated manganese transporter regulator MntR and/or inactivated binding site for MntR upstream of mntH1, wherein the MntH1 is preferably a polypeptide having at least 80%, at least 85%, at least 90%, at least 95%, at least 97%, at least 98% or 100% sequence identity with any one of the sequences of SEQ ID NO: 10.

In another preferred embodiments, the present application provides a lactobacillus strain, preferably L. kefiri, comprising the manganese transporter MntH1, inactivated manganese transporter regulator MntR and/or inactivated binding site for MntR upstream of mntH1, wherein the MntH1 is preferably a polypeptide having at least 80%, at least 85%, at least 90%, at least 95%, at least 97%, at least 98% or 100% sequence identity with any one of the sequences of SEQ ID NO: 11.

In another preferred embodiments, the present application provides a lactobacillus strain, preferably L. alimentarius, comprising the manganese transporter MntH1, inactivated manganese transporter regulator MntR and/or inactivated binding site for MntR upstream of mntH1, wherein the MntH1 is preferably a polypeptide having at least 80%, at least 85%, at least 90%, at least 95%, at least 97%, at least 98% or 100% sequence identity with any one of the sequences of SEQ ID NO: 12.

In another preferred embodiments, the present application provides a lactobacillus strain, preferably L. curvatus, comprising the manganese transporter MntH1, inactivated manganese transporter regulator MntR and/or inactivated binding site for MntR upstream of mntH1, wherein the MntH1 is preferably a polypeptide having at least 80%, at least 85%, at least 90%, at least 95%, at least 97%, at least 98% or 100% sequence identity with any one of the sequences of SEQ ID NO: 13.

In another preferred embodiments, the present application provides a lactobacillus strain, preferably L. zeae, comprising the manganese transporter MntH1, inactivated manganese transporter regulator MntR and/or inactivated binding site for MntR upstream of mntH1, wherein the MntH1 is preferably a polypeptide having at least 80%, at least 85%, at least 90%, at least 95%, at least 97%, at least 98% or 100% sequence identity with any one of the sequences of SEQ ID NO: 14.

In another preferred embodiments, the present application provides a lactobacillus strain, preferably L. kimchicus, comprising the manganese transporter MntH1, inactivated manganese transporter regulator MntR and/or inactivated binding site for MntR upstream of mntH1, wherein the MntH1 is preferably a polypeptide having at least 80%, at least 85%, at least 90%, at least 95%, at least 97%, at least 98% or 100% sequence identity with any one of the sequences of SEQ ID NO: 15.

Given that many Lactobacillus spp. are well-characterized, food-grade lactic acid bacterium (LAB) with generally recognized as safe (GRAS) status, the strains provided herein may be advantageously used as starter culture in the food industry. The present application provides compositions comprising Lactobacillus strains disclosed herein which can be used as starter culture. In the latter case, the composition may additionally comprise other starter bacteria for the fermentation of the food product. A skilled person in the art is able to select suitable starter bacteria based on the type of the food product. The present invention may be used in the preparation of food products including fermented food products, such as dairy products (including cheese), meat products or fermented dairy analogue or meat analogue products and other plant-based food products.

Manganese uptake activities can be measured using routine methods known in the art, see e.g. Kehres et al. “The NRAMP proteins of Salmonella typhimurium and Escherichia coli are selective manganese transporters involved in the response to reactive oxygen.” Molecular microbiology 36.5 (2000): 1085-1100. Alternatively, manganese scavenging activity may be determined via yeast inhibition assay described as following assay: The strains to be analyzed are grown in pasteurized cow milk for 24 hours at 37° C. Afterwards, two replicates of the fermented milk (150 μl) are transferred to a 96 microtiter plate and to half of the samples manganese is added to a final concentration of 6 mg/L. Afterwards, 50-100 CFU of D. hansenii (e.g. CHCC16374) per gram product are inoculated to the fermented milk with and without manganese addition, to determine if manganese is depleted. After 4 days of incubation at 17° C., a dilution row of the samples is spotted on selective YGC agar plates to analyze the yeast growth. The yeast growth can be enumerated by optical inspection. If differences between with or without manganese addition are observed, manganese scavenging from the tested strain is shown.

### Composition

In one aspect, the present application provides a composition, preferably a direct vat set composition, comprising the lactobacilli of the present invention. Advantageously, the bacteria may be supplied to the industry either as frozen or freeze-dried cultures for bulk starter propagation or as so-called “Direct Vat Set” (DVS) cultures, intended for direct inoculation into a fermentation vessel or vat for the production of a fermented product, such as a fermented dairy product like cheese. The starter culture composition is preferably in a frozen, dried or freeze-dried form, e.g. as a Direct Vat Set (DVS) culture. Preferably, the composition has a concentration of at least 10⁶ colony forming unit/g (cfu/g), such as at least 10⁷, at least 10⁸, at least 10⁹ or at least 10¹⁰ cfu/g.

However, the composition may also be a liquid that is obtained after suspension of the frozen, dried or freeze-dried cell concentrates in a liquid medium such as water or PBS buffer. Where the composition of the invention is a suspension, the concentration of viable cells is in the range of 10⁴ to 10¹² cfu (colony forming units) per ml of the composition including at least 10⁴ cfu per ml of the composition, such as at least 10⁵ cfu/ml, e.g. at least 10⁶ cfu/ml, such as at least 10⁶ cfu/ml, e.g. at least 10⁸ cfu/ml, such as at least 10⁹ cfu/ml, e.g. at least 10¹⁰ cfu/ml, such as at least 10¹¹ cfu/ml.

The composition of the present invention may additionally comprise cryoprotectants, lyoprotectants, antioxidants, nutrients, fillers, flavorants or mixtures thereof. The composition may be in frozen or freeze-dried form. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as cryoprotectants and lyoprotectant are known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate). Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

In preparing such compositions, it is preferably not to include too much manganese, because the bacteria may become less effective in inhibiting or delaying listeria growth when applied in the food product later, as described in WO2021/078764. Preferably, the composition comprises up to 600 ppm of manganese and wherein the concentration of the lactic acid bacteria colony forming unit/g of is at least 10⁶ colony forming unit/g (cfu/g), such as at least 10⁷, at least 10⁸, at least 10⁹ or at least 10¹⁰ cfu/g. In preferred embodiments, such products comprises 10-600 ppm of manganese, 30-600 ppm of manganese, 35-600 ppm of manganese, 40-600 ppm of manganese, 45-600 ppm of manganese, 50-600 ppm of manganese, 60-550 ppm of manganese, 100-500 ppm of manganese, 150-450 ppm of manganese, 190-400 ppm of manganese, 200-350 ppm of manganese, 250-300 ppm of manganese.

### Uses

In a further aspect, the manganese scavenging Lactobacillus strains or composition comprising the strains can be used to reduce free manganese and/or to inhibit or delay fungal (yeast and/mold) or listeria growth.

Since manganese is known to be important growth constraints for fungal growth, it is possible to use the bacteria disclosed herein to reduce the level of free manganese in the product. Free manganese concentration is preferably reduced to below about 0.01 ppm, such as below about 0.008 ppm, below about 0.006 ppm or below about 0.003 ppm. With such use a product in which unwanted yeast and or mold can hardly thrive can be obtained. It is envisioned that such spoilage prevention strategy is applicable even beyond food products and extending to other products which are generally prone to microbial contamination, such as feed products, cosmetic products, biologic products, health care products, pharmaceutical products and the like.

Furthermore, it is known that listeria growth can also be inhibited for delayed by manganese depletion (van Gijtenbeek et al. 2021). Therefore, food safety by controlling growth of Listeria during the shelf life of food products may be ensured, using the lactobacilli of the present invention.

“Free manganese” or sometimes “manganese” in accordance with the present application refers to manganese which is present in a product (i.e. forming part of product, such as within the product or on the surface of a product) that is available to be taken up by fungi, including yeasts and molds, or other bacteria. For example, free manganese refers to the manganese that is present in the matrix of the product.

Preferably, the use is carried out in the applied product in the presence of glucose. The inventors have surprisingly found that the manganese scavenging activity is increased in the presence of glucose. In preferred embodiments, the use is carried out in the presence of at least 0.2 g/L glucose in the product, such at least 0.5 g/L glucose, such at least 1.0 g/L glucose, such at least 2.0 g/L glucose, such at least 3.0 g/L glucose, such at least 4.0 g/L glucose, such at least 5.0 g/L glucose

In general, inhibiting means a decrease, whether partial or whole, in function and activity of cells or microorganisms. As used herein, the terms “to inhibit” and “inhibiting” in relation to the microorganism mean that the growth, the number, or the concentration of a given microorganism is the same or reduced. This can be measured by any methods known in the field of microbiology. Inhibition can be observed by comparing the growth, number or concentration in or on a product with reduced free manganese to a control. The control can be the same product but without reduced free manganese. The term “to delay” in general means the act of stopping, postponing, hindering, or causing something to occur more slowly than normal. As used herein, “delaying growth” of a microorganism refers to the act of postponing the growth of said microorganism. This can be observed by comparing the time needed for the microorganism to grow to a given level in two products, one of which with reduced manganese and the other one without (but otherwise the same). In some embodiments, “delaying growth” refers to delaying by 7 days or more.

Fungal or listeria growth can be measured with various methods known to a skilled person in the art. For example, fungal growth can be measured by density or size of colony, cell number, mycelial mass changes, spore production, hyphal growth, colony-forming units (CFU) and the like, depending on the fungus type and the product to which the method is applied. Fungal growth can also be observed by measuring the change in nutrient or metabolite concentrations, such as carbon dioxide release and oxygen uptake. Listeria growth may also be determined using routine enumeration methods known in the art. One may apply standard protocols in US FDA's Bacteriological Analytical Manual (BAM) (Hitchins et al., “BAM: Detection and Enumeration of Listeria monocytogenes.” Bacteriological analytical manual (2016)) or protocols published by the European and International Standard method EN ISO 11290-1:2017 (ISO, PNEN. “11290-1: 2017. Microbiology of the food chain—Horizontal method for the detection and enumeration of Listeria monocytogenes and of Listeria spp.”). Other methods can also be used, such as described in Law et al. “An insight into the isolation, enumeration, and molecular detection of Listeria monocytogenes in food.” Frontiers in microbiology 6 (2015): 1227.

Furthermore, the present application provides use of one or more manganese scavenging Lactobacillus strains or composition described herein to prepare a fermented food product. Such food product is preferably fermented dairy or dairy analogue products, including yogurt, cheese and corresponding analogue products.

“Dairy product” includes, in addition to milk, products derived from milk, such as cream, ice cream, butter, cheese and yogurt, as well as secondary products such as lactoserum and casein and any prepared food containing milk or milk constituents as the main ingredient, such as formula milk. In one preferred embodiment, the dairy product is a fermented dairy product. Milk is generally understood as the lacteal secretion obtained by milking any mammal, such as cows, sheep, goats, buffaloes or camels. In a preferred embodiment, the milk is cow's milk.

Dairy or meat analogue products refer to dairy-like or meat-like products, which are products used as culinary replacements for dairy or meat products, prepared where one or more animal constituents have been replaced with other ingredients and the resulting food resembles the original product. “Dairy analogue product” includes products derived from plant-based milk such as soy milk. For the purpose of the present application, the term “milk” should be understood as to include protein/fat solutions made of plant materials, e.g. soy milk.

### Methods of Reducing Free Manganese

In a further aspect, the present application provides a method of reducing free manganese in a product, such as food product including fermented food product, comprising the steps of
selecting one or more manganese scavenging Lactobacillus strains that comprises a manganese transporter MntH1, characterized in that the strain comprises inactivated MntR and/or inactivated binding site for MntR upstream of mntH1,
adding the Lactobacillus strain(s), preferably as a Direct Vat Set (DVS) culture composition, to the product.

When applying to food products, the method may further comprise the step of fermenting said food product to a target pH. The manganese scavenging activity may lead to the inhibition or delay of the growth of unwanted microorganisms, such as yeast, mold and/or listeria.

It is preferred that the manganese in the product is reduced to a concentration of below about 0.01 ppm, preferably below about 0.008 ppm, or below about 0.006 ppm, preferably below about 0.005 ppm, below about 0.004 ppm, below about 0.003 ppm, below about 0.002 ppm or below about 0.001 ppm.

In one preferred embodiment, the present application is directed to a method of inhibiting or delaying growth of fungi in a food product, comprising reducing free manganese concentration in a food matrix of the food product. As used herein, the term “food matrix” refers to the food's composition and structure. It is based on the concept that nutrients are contained in a continuous medium.

The term “reduce” or “reducing” generally means lowering the amount of a substance in a given context. As used herein, the term “to reduce free manganese” or “reducing free manganese” means to reduce the amount of manganese present in a product that is available to be taken up by fungi, including yeasts and molds.

For example, this can be carried out by removing manganese present in the product or in a material which is to become part of the product. For example, this can be carried out by subjecting the raw material ion exchange chromatography to remove manganese so that the concentration in the final product is reduced.

Once having access, fungi rapidly colonize, increase in population and take up nutrients from their immediate surroundings. In some embodiments, given that fungi may first come into contact with a product on the surface, it is within the spirit of the present invention that the step of reducing is carried out on parts of the product, for example in the exterior part of the product such as the coating or an outer layer. In such cases, the reducing step nevertheless leads to an overall decrease in the concentration in the product.

Manganese concentration or manganese level as used herein is expressed in parts per million (“ppm”) calculated on a weight/weight basis. Reducing free manganese in a product to a concentration below a value means reducing free manganese in the product or parts thereof such that the concentration of free manganese in the entire product by weight is reduced. Methods of determining trace elements such as manganese are known in the art and described for example in Nielsen, S. Suzanne, ed. Food analysis. Vol. 86. Gaithersburg, MD: Aspen Publishers, 1998.

As used herein, the term “about” indicates that values slightly outside the cited values, i.e., plus or minus 0.1% to 10%. Thus, concentrations slightly outside the cited ranges are also encompassed by the scope of the present inventions.

Methods of measuring of manganese at low concentration are well known to a person skilled in the art. Such methods include atomic absorption spectroscopy, atomic emission spectroscopy, mass spectrometry, neutron activation analysis and x-ray fluorimetry (see e.g., Williams et al. “Toxicological profile for manganese.” (2012)).

In one embodiment, the method is used to inhibit the growth of yeast, such as Candida spp., Meyerozyma spp., Kluyveromyces spp., Pichia spp., Galactomyces spp., Trichosporon spp., Sporidiobolus spp., Torulaspora spp., Cryptococcus spp., Sacharomyces spp., Yarrowia spp., Debaryomyces spp., and Rhodoturola spp. Preferably, the fungi is a yeast selected from the group consisting of Torulaspora spp., Cryptococcus spp., Sacharomyces spp., Yarrowia spp., Debaryomyces spp., Candida spp. and Rhodoturola spp. More preferably, the fungus is a yeast selected from the group consisting of Torulaspora delbrueckii, Cryptococcus fragicola, Sacharomyces cerevisiae, Yarrowia lipolytica, Debaryomyces hansenii and Rhodoturola mucilaginosa.

In one embodiment, the method is used to inhibit the growth of mold. Preferably, the fungus is a mold selected from the group consisting of Aspergillus spp., Cladosporium spp., Didymella spp. or Penicillium spp. More preferably, the fungus is a mold selected from the group consisting of Penicillium brevicompactum, Penicillium crustosum, Penicillium solitum, Penicillium carneum, Penicillium paneum, and Penicillium roqueforti.

In one embodiment, the method is used to inhibit the growth of Listeria. The genus Listeria as of 2019 is known to contain 20 species: L. aquatica, L. booriae, L. cornellensis, L. costaricensis, L. goaensis, L. fleischmannii, L. floridensis, L. grandensis, L. grayi, L. innocua, L. ivanovii, L. marthii, L. monocytogenes, L. newyorkensis, L. riparia, L. rocourtiae, L. seeligeri, L. thailandensis, L. weihenstephanensis, and L. welshimeri. Two well-known species are Listeria monocytogenes or Listeria innocua. L. innocua and L. listeria have been found to behave similarly in dairy environment. Listeria innocua is generally considered nonpathogenic and is used as surrogate in pilot studies which reflect and predict inhibition of Listeria monocytogenes. In addition, a fatal case of Listeria innocua bacteremia has been reported (Perrin et al, Journal of Clinical Microbiology 41.11 (2003): 5308-5309). Preferably, the method is used to inhibit the growth of Listeria monocytogenes.

When measuring free manganese, such free manganese does not include the manganese which is found intracellularly. Rather, free manganese refers to the manganese that is found extracellularly, i.e. in the cell-free parts of the product, since they would be available to be taken up by other microorganism like yeast, mold or other bacteria. Thus, in such cases, concentration of free manganese should be measured taking only extracellular manganese into account. This can be done for example by removing cells (such as starter cultures) by centrifugation and obtaining cell-free supernatant, followed by measuring the manganese in the cell-free supernatant.

As used herein, the term “bacteria strain” or “strain” has its common meaning in the field of microbiology and refers to a genetic variant of a bacterium.

When applying the present methods, one skilled in the art may first determine the manganese level which is present in the products to be treated, and then determine accordingly the amount of the lactobacilli to be applied. Manganese concentration for food products is well studied and can be found in national food composition databases such as Danish Food Composition Databank and Canadian Nutrient Files. In general, manganese is present at a concentration of at least 0.03 ppm for milk, making dairy products susceptible for fungal or listeria contamination. Manganese levels have been reported to range from 0.04 to 0.1 ppm in cow milk and up to 0.18 ppm in goat or sheep milk (Muehlhoff et al., Milk and dairy products in human nutrition. Food and Agriculture Organization of the United Nations (FAO), 2013). As for fermented dairy products like cheese, the manganese level usually increases due to the concentration process from milk, often up to 10-fold or more. Different levels have been reported for various types of cheeses, for example about 0.06 ppm for ricotta cheese, 0.11 ppm for cream cheese, 0.34 ppm for brie, 0.3 ppm for mozzarella, 0.7 ppm for cottage cheese, 0.68 ppm for gouda and 0.74 ppm for cheddar cheese (Smit, L. E., et al. The nutritional content of South African cheeses. ARC-Animal Improvement Institute, 1998; Gebhardt, Susan, et al. “USDA national nutrient database for standard reference, release 12.” United States Department of Agriculture, Agricultural Research Service, 1998). Higher manganese levels are found in plant materials.

Manganese concentration can be measured according the standard procedure as described in “Foodstuffs—Determination of trace elements—Pressure digestion” in European Standard EN13805:2014 published by European Committee for Standardization or as described in “Water quality—Determination of selected elements by inductively coupled plasma optical emission spectrometry (ICP-OES)” in ISO 11885:2007 published by International Organization for Standardization.

### Products

The present invention also provides products comprising the manganese scavenging Lactobacillus strain(s) or compositions described herein. In some embodiments, the product is a food product, feed product, cosmetic product, health care product or a pharmaceutical product. “Food” and “food product” have the common meaning of these terms. “Food product” and “feed product” refer to any products suitable for consumption by humans or animals. Such products can be fresh or perishable food products as well as stored or processed food products. Food products include, but are not limited to, fruits and vegetables including derived products, grain and grain-derived products, dairy products, meat, poultry and seafood. More preferably, the food product is a meat product or dairy products, such as yogurt, tvarog, sour cream, cheese and the like. The food product typically has a pH of about 3.5 to about 6.5, such as about 4 to about 6, such as about 4.5 to about 5.5, such as about 5.

The main food categories prone to fungal or listeria spoilage are dairy products having intermediate to high water activity, such as yogurt, cream, butter, cheese and the like. However, it is also envisioned that the present invention is suitable for food products having lower water activities, such processed meat, cereals, nuts, spices, dried milk, dried meats and fermented meat.

Of note, manganese can be found naturally in many food sources including leafy vegetables, nuts, grains and animal products. Typical ranges of manganese concentrations in common foods are for example 0.4-40 ppm in grain products, 0.1-4 ppm in meat, poultry, fish and eggs, 0.4-7 ppm in vegetable products. Concentration of manganese varies in milk, depending on the animal from which it is produced, the feed, as well as the season. In general, manganese is present at a concentration of at least 0.03 ppm in dairy products, for example 0.08 ppm for skimmed milk, and 0.1 ppm or higher for whole milk. With the present finding of the inventors, reducing the manganese amount in such products or products prepared therefrom would render them more resistant to spoilage.

The present invention is particularly useful in inhibiting or delaying growth of fungi in dairy products. In such products, contamination with yeast and molds are common and limits the shelf life of such products.

### Method of Preparing Dairy or Dairy Analogue Products

The methods disclosed herein are particularly useful to inhibit or delay yeast, mold and/or listeria growth in fermented dairy or dairy analogue products.

The expression “fermented dairy product” means a product wherein the preparation involves fermentation of a milk base with a lactic acid bacterium. “Fermented dairy product” as used herein includes but is not limited to products such as thermophilic products (e.g. yogurt) and mesophilic products (e.g. sour cream).

In a preferred embodiment, fermented food product is selected from the group consisting of quark, cream cheese, fromage frais, greek yogurt, skyr, labneh, butter milk, sour cream, sour milk, cultured milk, kefir, lassi, ayran, twarog, doogh, smetana, yakult and dahi.

In another preferred embodiment, fermented food product is a cheese, including continental type cheese, fresh cheese, soft cheese, cheddar, mascarpone, pasta filata, mozzarella, provolone, white brine cheese, pizza cheese, feta, brie, camembert, cottage cheese, Edam, Gouda, Tilsiter, Havarti or Emmental, Swiss cheese, and Maasdamer.

The manganese transporter is not present in L. delbrueckii subsp. bulgaricus and only displays low expression in Streptococcus thermophilus, the two strains found in the starter culture in yogurt, making them particularly susceptible to fungal spoilage. It is therefore preferable to include the Lactobacillus strain(s) of the present invention to scavenge free manganese present in yogurt.

The term “yogurt” has its usual meaning and is generally defined in accordance with relevant official regulations and standards are well known in the field. Starter cultures used for making yogurt comprises at least one Lactobacillus delbrueckii subsp. bulgaricus strain and at least one Streptococcus thermophilus strain. A skilled person is able to select a suitable starter culture for preparing the intended products.

A food substrate is provided as starting material. To make fermented dairy products, the food substrate is a milk base which can optionally be plant based.

“Milk base” is broadly used in the present application to refer to a composition based on milk or milk components which can be used as a medium for growth and fermentation of a starter culture.

Milk bases include, but are not limited to, solutions/suspensions of any milk or milk like products comprising protein, such as whole or low-fat milk, skim milk, buttermilk, reconstituted milk powder, condensed milk, dried milk. It may be prepared from plant material.

Milk base, if containing lactose, may also be lactose-reduced depending on the need of the consumers. Lactose-reduced milk can be produced according to any method known in the art, including hydrolyzing the lactose by lactase enzyme to glucose, or by nanofiltration, electrodialysis, ion exchange chromatography and centrifugation.

To ferment the milk base, a starter culture is added. The term “starter culture” as used in the present context refers to a culture of one or more food-grade microorganisms in particular lactic acid bacteria, which are responsible for the acidification of the milk base.

The skilled person is able to adjust various parameters such as pH, temperature, oxygen, addition of carbohydrates, and amount of starter culture as well as manganese scavenging bacteria to achieve the desired results, taking into consideration the properties of the food product such as water activity, nutrients, level of naturally occurring manganese, shelf life, storage conditions, packing, etc.

Manganese scavenging bacteria may be added before, at the start, or during the fermentation. Depending on parameters chosen, the fermentation may take several hours, such as at least 5 hours, such as at least 10 hours, such as at least 15 hours, such as at least 20 hours, such as at least 1 day, 2 days, 3 days or more. In some embodiments, the fermentation takes from three, four, five, six hours or longer.

These conditions include the setting of a temperature which is suitable for the particular starter culture strains. For example, when the starter culture comprises mesophilic lactic bacteria, the temperature can be set to about 30° C., and if the culture comprises thermophilic lactic acid bacterial strains, the temperature is kept in the range of about 35° C. to 50° C., such as 40° C. to 45° C. The setting of the fermentation temperature also depends on the enzyme(s) added to the fermentation which can be readily determined by a person of ordinary skill in the art. In a particular embodiment of the invention the fermentation temperature is between 35° C. and 45° C., preferably between 37° C. and 43° C., and more preferably between 40° C. and 43° C. In another embodiment, the fermentation temperature is between 15° C. and 35° C., preferably between 20° C. and 35° C., and more preferably between 30° C. and 35° C.

Fermentation can be terminated using any methods known to in the art. In general, depending on various parameters of the process, the fermentation can be terminated by making the milk base unsuitable for the strain(s) of the starter culture to grow. For example, termination can be carried out by rapid cooling of the fermented product when a target pH is reached. It is known that during fermentation acidification occurs, which leads to the formation of a three-dimensional network consisting of clusters and chains of caseins. The term “target pH” means the pH at which the fermentation step ends. The target pH depends on the fermented product to be obtained and can be readily determined by a person of ordinary skill in the art.

In a particular embodiment of the invention, fermentation is carried out until at least a pH of 5.2 is reached, such as until a pH of 5.1, 5.0, 4.9, 4.8, 4.7, 4.6, 4.5, 4.4, 4.3, 4.2, 4.1, 4.0, 3.9, 3.8 or 3.7 is reached. Preferably, the fermentation is carried out until a target pH between 4.0 and 5.0 and more preferably between 4.0 and 4.6 is reached. In a preferred embodiment, the fermentation is carried out until target pH below 4.6 is reached.

In a further embodiment, the method further comprises packing the food product to reduce contact with unwanted microorganisms such as yeast or mold. It is also preferred to store the product under cold temperature (below 15° C.) to help extend shelf life.

Included in the present application is a food product obtained by the methods described herein. The product obtained by the present application is preferably a product, including fermented dairy or dairy analogue product with a concentration of free manganese reduced to less than 0.01 ppm after being stored for at least two days, for example at least 3 days, at least 4 days, more preferably at least 5 days, at least 6 days, at least 7 days, at least 8 days, at least 9 days, at least 10 days, at least 11 days, at least 12 days, at least 13 days, and at least 14 days.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### Deposit and Expert Solution

The applicant requests that a sample of the deposited microorganisms stated below may only be made available to an expert, subject to available provisions governed by Industrial Property Offices of States Party to the Budapest Treaty, until the date on which the patent is granted.

The applicant deposited the Lactobacillus paracasei strain CHCC14676 on 2012 Feb. 2 at Leibniz Institute DSMZ—German Collection of Microorganisms and Cell Cultures, Inhoffenstr. 7B, D-38124 Braunschweig and received the accession No.: DSM 25612.

The applicant deposited the Lactobacillus paracasei strain CHCC15860 on 2015 Jul. 16 at Leibniz Institute DSMZ—German Collection of Microorganisms and Cell Cultures, Inhoffenstr. 7B, D-38124 Braunschweig and received the accession No.: DSM 32092.

### EXAMPLES

### Example 1 Lactobacillus paracasei with Inactivated MntR

### Construction of LpMntR

L. paracasei strain CHCC14676 (deposited as DSM 25612) was used as mother strain. It expresses the manganese transporter MntH1 sequence as set forth in SEQ ID NO: 2 and has the MntR sequence as set forth in SEQ ID NO: 31. The binding site sequence for MntR upstream of mntH1 is as set forth in SEQ ID NO: 20.

A clean mntR knockout strain was constructed from the mother strain via a double crossover strategy with non-replicating plasmid pCS1966 and oroP/5-FOA-based counter-selection for plasmid curing. Flanks were amplified from genomic DNA including 1000 bp upstream (using primer pair EFB0195+EFB0196) and downstream (using primer pair EFB0197+EFB0198) of the MntR gene, respectively. All fragments were gel purified and an overlap PCR was performed with the primer pair EFB0195+EFB0198 to fuse the two constructs. The plasmid backbone was amplified in two fragments with primers EFB0122+EFB0123 and EFB0124+EFB0125 and a Gibson assembly was performed to fuse the three remaining fragments to form the final plasmid pEBF051, which was transformed to L. lactis. This plasmid was afterwards transformed into L. paracasei and integrants were obtained on selective agar plates. The integrant was afterwards cultivated and plated on counter selective plates and the presence of wild type revertant and clean knock-out mutants were analyzed by PCR and confirmed by sequencing.

TABLE 5
Sequences used in this example

| SEQ ID NO | | Sequence (5′→3′) |
|---|---|---|
| 45 | EFB0122 | GCTTATCGATACCGTCGACCTCGAG |
| 46 | EFB0123 | GTCGTTAAATGCCCTTTACCTGTTCCAATTTCGTAAACGGTATCGGTTTC |
| 47 | EFB0124 | GAAACCGATACCGTTTACGAAATTGGAACAGGTAAAGGGCATTTAACGAC |
| 48 | EFB0125 | CGGGGGATCCACTAGTTCTAGAGCGGC |
| 49 | EFB0195 | GCCGCTCTAGAACTAGTGGATCCCCCGGAATCTGAATAAGACAAAGCTTG |
| 50 | EFB0196 | CCCTGCTTTCTCTAGACTTAATTTACATCCTTACTTTTAATTTG |
| 51 | EFB0197 | CAAATTAAAAGTAAGGATGTAAATTAAGTCTAGAGAAAGCAGGG |
| 52 | EFB0198 | CTCGAGGTCGACGGTATCGATAAGCCATAATTTTGCCTGCGACAAAAG |

### Influence of Manganese Addition

The influence of manganese addition to milk on acidification behavior of the LpMntR and the mother strain DSM 25612 is evaluated. The acidification of the strains in milk was measured as an indicator for growth.

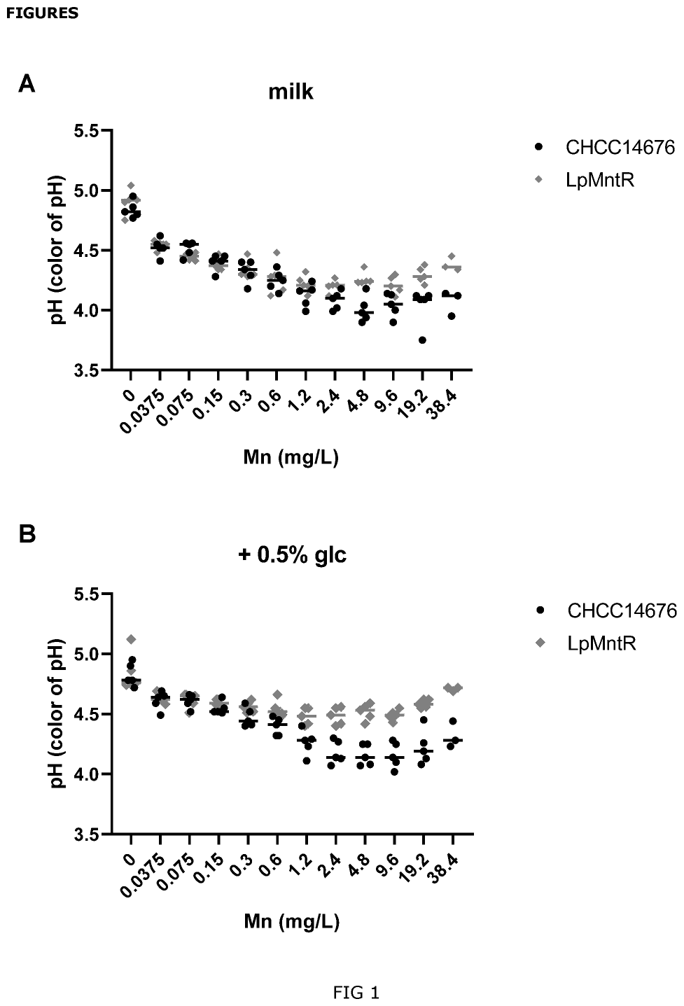

Both strains were grown with different manganese concentrations ranging from 0 to 38 mg/L in 2 ml of milk (FIG. 1A) and milk supplemented with 0.5% glucose (FIG. 1B) in a 96 deep-well plate. The plate was incubated at 37° C. overnight and the pH was measured by the color change of a pH indicator as previously described in Poulsen et al. 2019 (Poulsen, V. K., Derkx, P., Oregaard, G. (2019): “High-Throughput Screening for Texturing Lactococcus Strains”. FEMS Microbiological Letters), where color (hue) values were calibrated to pH values.

Upon the addition of 0.0375-0.6 mg/L manganese, no difference in growth was detected between the two strains. This shows that growth differences are not the basis for mntH1 expression or improved yeast inhibition against yeast as shown in Example 2-3. However, at higher concentrations, the mother strain was able to acidify to lower pH values compared to LpMntR.

It is surprising that when high manganese was present, LpMntR remains to acidify. This is in contrast to B. subtilis (Que et al. 2000) where a deletion of mntR resulted in a strain sensitive to elevated manganese concentration.

### Example 2 Expression of MntH1 in DSM 25612 and LpMntR

MntH1 is an important manganese transport protein in the mother strain which takes up manganese. The expression strength of the mntH1 gene in the L. paracasei mother strain DSM and its mntR deletion mutant (LpMntR) was analyzed by a plasmid based promoter fusion with a fluorescent protein.

The mntH1 promoter was cloned in front of a red fluorescent protein. First, the gene sequence for mCherry (GenBank ID AY678264, (Shaner et al., 2004)) was codon-optimized for low-GC LAB using Optimizer (Puigbò et al., 2007) with the ‘guided random’ and ‘Codon usage (HEG)’ settings for the L. casei type strain ATCC334. The P11 promoter is a strong constitutive synthetic promoter developed in L. plantarum and its sequence was used as originally described (Rud et al., 2006). The combined P11 promoter and optimized mCherry gene sequence was ordered as a synthetic construct (GenScript, Piscataway, NJ, USA) and subsequently cloned into the broad host range vector pNZ8148 (MoBiTec, Goettingen, Germany). P11-mCherry was amplified from the GenScript vector using primers EFB0057+EFB0060 while the pNZ8148 backbone was amplified with primers EFB0061+0062 following by Gibson assembly. Afterwards, the nisA promoter present on pNZ8148 was replaced by the mntH1 promoter. For this, the mntH1 promoter was amplified using primers EFB0185+EFB0186 and the backbone plasmid with the primers EFB0180+EFB0181, followed by Gibson assembly resulting in plasmid pEFB045.

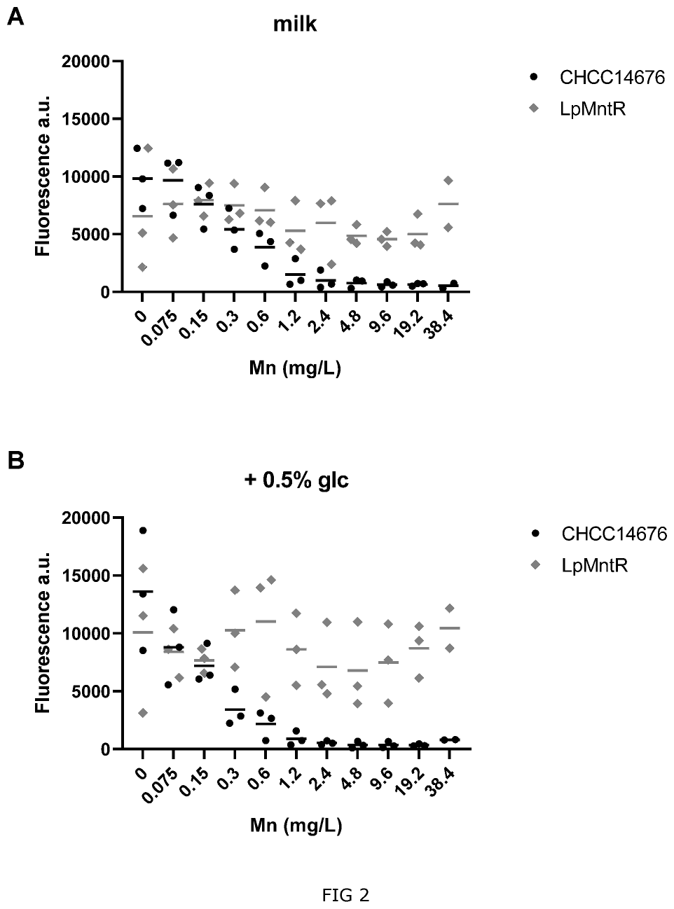

This plasmid was introduced by electroporation both into the mother strain and the mntR deletion mutant (LpMntR). Afterwards the strains were grown in different manganese concentrations ranging from 0 to 38 mg/L in 2 ml milk (FIG. 2A) and milk supplemented with 0.5% glucose (FIG. 2B) in a 2 ml deep well plate. Milk naturally contains about 0.06 mg/L manganese. The plate was incubated at 37° C. overnight and 100 μl aliquot was transferred to a 96 low well plate. After another day of incubation at room temperature the fluorescence was measured with excitation at 579 nm and emission at 616 nm in a plate reader.

In the mother strain, a decreased of the mntH1 expression upon addition of manganese was seen, with a complete repression of its transcription when more than 1.2 mg/L manganese was added This shows that expression of mntH1 is abolished completely at higher manganese concentrations. In contrast, the expression of mntH1 in LpMntR stayed constantly high in all conditions. This shows that MntR is responsible for repressing the expression of the mntH1 gene in the presence of manganese.

TABLE 6
Sequences used in this example

| SEQ ID NO | | Sequence (5′→3′) |
|---|---|---|
| 53 | EFB0057 | GAAGAAGGTTTTTATATTACAGCTCCAGATCTAGCGCTATAGTTGTTGACAG |
| 54 | EFB0060 | CTTGGTTTTCTAATTTTGGTTCAAAGAAAGCTTTTATTTGTACAGCTCATCC |
| 55 | EFB0061 | GGATGAGCTGTACAAATAAAAGCTTTCTTTGAACCAAAATTAGAAAACCAAG |
| 56 | EFB0062 | CTGTCAACAACTATAGCGCTAGATCTGGAGCTGTAATATAAAAACCTTCTTC |
| 57 | EFB0180 | TCTAGAGTGAGTAAAGGCGAAG |
| 58 | EFB0181 | AGATCTGGAGCTGTAATATAAAAACC |
| 59 | EFB0185 | TCTTCTTCGCCTTTACTCACAATAACTCTCCCCTTTCGTTTG |
| 60 | EFB0186 | GGTTTTTATATTACAGCTCCAGATCTGTGACTTTTTAACAATAACG |

### Example 3 Yeast Inhibition of DSM 25612 and LpMntR

Manganese scavenging activity of LpMntR was compared to its mother strain DSM 25612 as well as the influence of addition of manganese. It is known that low manganese concentrations are the major limitation for yeast growth (WO2019/202003). Therefore, yeast inhibition reflects the manganese scavenging activity of the strains. In the experiments, addition of manganese is expected to restore the growth of yeast and shows that it is the limiting factor for yeast growth.

The individual Lactobacillus strains were grown in MRS overnight. 10 μl of the preculture was used to inoculated 2 ml milk (which has 0.06 mg/L manganese) with or without 0.5% glucose both supplemented with a manganese gradient ranging from 0-0.6 mg/L. The milk was fermented at 37° C. overnight and next day 150 μl of the fermented milk was transferred to individual wells in a 96 well plate. All the wells were inoculated with about 20 CFUs of Debaryomyces hansenii (Chr. Hansen culture collection, CHCC16374). After 5 days a 1000-fold dilution was spotted on selective YGC plates to analyze the yeast growth.

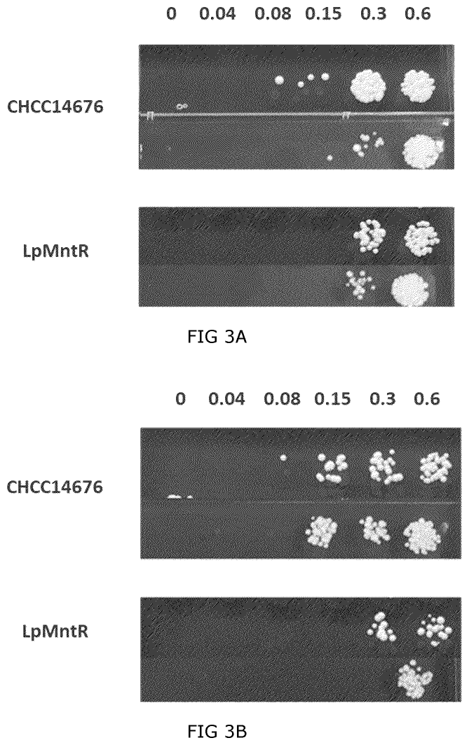

FIG. 3A depicts inhibition of the yeast under various manganese addition without addition of glucose. While the mother strain can only inhibit the yeast growth upon concentrations of 0.08 mg/L manganese, LpMntR could inhibit the yeast growth upon 0.15-0.3 mg/L manganese addition. This demonstrates that the MntR inactivated strain is applicable in a broader application range where higher manganese concentrations are present.

FIG. 3B depicts inhibition of the yeast under various manganese addition with addition of glucose. Surprisingly, in the presence of glucose, the MntR inactivated strain exhibited higher manganese scavenging activity and thus yeast inhibition.

### Example 4 Lactobacillus rhamnosus with Inactivated MntR

### Construction of LrMntR

L. rhamnosus strain CHCC15860 (deposited as DSM 32092) was used as mother strain. It expresses the manganese transporter MntH1 sequence as set forth in SEQ ID NO: 4 and has the MntR sequence as set forth in SEQ ID NO: 33. The binding site sequence for MntR upstream of mntH1 is as set forth in SEQ ID NO: 25.

A clean mntR knockout strain was constructed from the mother strain via a double crossover strategy with non-replicating plasmid pCS1966. Flanks were amplified from genomic DNA including 1000 bp upstream (using primer pair AMB546+AMB547) and downstream (using primer pair AMB548+AMB549) of the MntR gene, respectively. The plasmid backbone was amplified in one fragment with primers AMB550+AMB551. All three fragments were gel purified and a Gibson assembly was performed to fuse the three fragments to form the final plasmid pAMB058, which was transformed to L. lactis. This plasmid was afterwards transformed into L. rhamnosus and integrants were obtained on selective agar plates. The integrant strain was afterwards made competent and transformed with a targeting plasmid pAMB060, which was based on a low-copy replicating plasmid pIL252. The targeting plasmid contained MAD7 nuclease expressed from p5 promoter, and a gRNA cassette consisting of p32 promoter cloned until the TSS site, the gRNA repeat and a spacer targeting the MntR gene (ACAGTGTAATCAATCAATGAA). The targeting plasmid was cloned in two parts from another CRISPR-MAD7 targeting plasmid (pAMB054), where only the spacer sequence was exchanged by being added to the primers as overhangs for Gibson assembly. The two fragments were amplified using the primer pairs AMB460+AMB556 and AMB557+AMB463, then both were gel purified and fused using Gibson assembly, which was followed by a transformation into L. lactis. The transformation of the integrant strain with the targeting plasmid pAMB060 resulted in obtaining an mntR deletion mutant. The mutant was then further grown overnight in non-selective conditions in order to lose the targeting plasmid, which resulted in a clean mntR knockout strain.

TABLE 7
Sequences used in this example

| SEQ ID NO | | Sequence (5′→3′) |
|---|---|---|
| 61 | AMB546 | AGTGGATCCCCCGCCGAAGCTGAATAAGACC |
| 62 | AMB547 | CCCAAGTCTTGATTTACATCCTTACTTTTAATTTGC |
| 63 | AMB548 | GTAAGGATGTAAATCAAGACTTGGGAAAGCAG |
| 64 | AMB549 | GGTATCGATAAGCAAAATCTTGCCTGCGACAAAAG |
| 65 | AMB550 | AGGCAAGATTTTGCTTATCGATACCGTCGAC |
| 66 | AMB551 | ATTCAGCTTCGGCGGGGGATCCACTAGTTC |
| 67 | AMB460 | AAGTGAGGGAAAGGCTACTAAAACGTCGAGG |
| 68 | AMB556 | TTATAATCCATGGTTCATTGATTGATTACACTGTATCTACAAGAGTAGAAATTAAAAAGG |
| 69 | AMB557 | TACTCTTGTAGATACAGTGTAATCAATCAATGAACCATGGATTATAAAGAGAGCGGC |
| 70 | AMB463 | CGTTTTAGTAGCCTTTCCCTCACTTCGTTC |

### Influence of Manganese Addition on Acidification

The influence of manganese addition to milk on acidification behavior of the LrMntR and the mother strain CHCC15860 is evaluated. The acidification curve of the strains in milk was measured and followed as an indicator for growth.

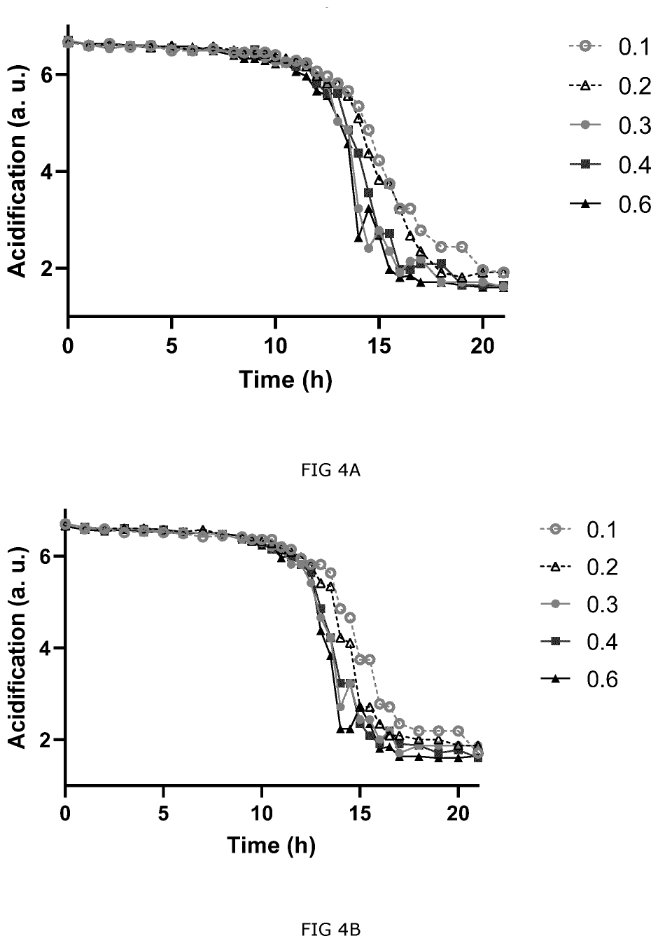

Both strains were grown with different manganese concentrations ranging from 0.1 to 0.6 mg/L in 2 ml of milk in a 96 deep-well plate. The plate was incubated at 37° C. overnight and the pH was measured by the color change of a pH indicator as previously described in Poulsen et al. 2019 (Poulsen, V. K., Derkx, P., Oregaard, G. (2019): “High-Throughput Screening for Texturing Lactococcus Strains”. FEMS Microbiological Letters), where color (hue) values were calibrated to pH values. The acidification for CHCC15860 and LrMntR are shown in FIG. 4A and FIG. 4B, respectively.

No significant differences in growth were detected between the two strains under different manganese addition. This shows that growth differences were not the basis for improved inhibition against yeast shown in Example 5.

### Example 5 Yeast Inhibition of CHCC15860 and LrMntR

The individual Lactobacillus strains were grown in MRS overnight. 10 μl of the preculture was used to inoculated 2 ml milk (which has 0.06 mg/L manganese) supplemented with a manganese gradient ranging from 0-0.6 mg/L. The milk was fermented at 37° C. overnight and next day 150 μl of the fermented milk was transferred to individual wells in a 96 well plate. All the wells were inoculated with about 20 CFUs of Debaryomyces hansenii (Chr. Hansen culture collection, CHCC16374). After 4 days a 100-fold dilution was spotted on selective YGC plates to analyze the yeast growth.

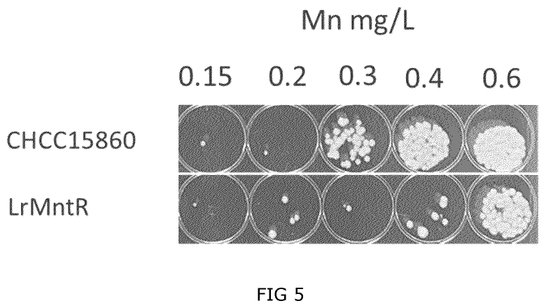

FIG. 5 depicts inhibition of the yeast under various manganese addition. While the mother strain can only inhibit the yeast growth upon concentrations of <0.3 mg/L manganese, LrMntR could inhibit the yeast growth upon 0.4 mg/L manganese addition.

The foregoing examples demonstrate that MntR inactivated strains from different lactobacilli species are applicable in a broader application range where higher manganese concentrations are present.

1. A method of improving manganese scavenging activity in a lactobacillus strain, comprising:
providing one or more Lactobacillus strains which express the manganese transporter MntH1 as the mother strain,
obtaining one or more mutants from the mother strain, in which the manganese transporter regulator MntR or its binding site upstream of mntH1 is inactivated, preferably by substitution, truncation, deletion, point mutation and/or knock-out, and
selecting from the obtained mutants one or more daughter Lactobacillus strains having higher manganese scavenging activity compared to the mother strain.

2. The method according to claim 1, wherein the Lactobacillus as mother strain is preferably L. salivarius, L. reuteri, L. brevis, L. kefiri, L. alimentarius, L. zeae or L. kimchicus, and more preferably L. curvatus, L. sakei, L. casei, L. paracasei, L. rhamnosus, L. plantarum or L. fermentum.

3. The method according to any one of the preceding claims, wherein the MntH1 sequence is a polypeptide having at least 60%, at least 65%, at least 70%, at least 75%, at least 80%, at least 85%, at least 90%, at least 95%, at least 97%, at least 98% or 100% sequence identity with any one of the sequences of SEQ ID NO: 1-15, preferably with SEQ ID NO: 1 or 2.

4. The method according to any one of the preceding claims, wherein the daughter Lactobacillus strains has higher manganese scavenging activity compared to the mother strain in milk having a manganese concentration of 0.135 mg/L.

5. A lactobacillus strain comprising a manganese transporter MntH1, characterized in that the strain comprises inactivated manganese transporter regulator MntR and/or inactivated binding site for MntR upstream of mntH1.

6. The lactobacillus strain according to claim 5, wherein the bacteria strain is preferably L. salivarius, L. reuteri, L. brevis, L. kefiri, L. alimentarius, L. zeae or L. kimchicus, and more preferably L. curvatus, L. sakei, L. casei, L. paracasei, L. rhamnosus, L. plantarum or L. fermentum.

7. The lactobacillus strain according to claims 5-6, wherein the MntH1 is a polypeptide having at least 60%, at least 65%, at least 70%, at least 75%, at least 80%, at least 85%, at least 90%, at least 95%, at least 97%, at least 98% or 100% sequence identity with any one of the sequences of SEQ ID NO: 1-15, preferably with SEQ ID NO: 1 or 2.

8. A composition comprising any one of the bacterial according to claims 5-7.

9. The composition according to claim 8, preferably a direct vat set composition with a concentration of at least 10⁶ colony forming unit/g (cfu/g), such as at least 10⁷, at least 10⁸, at least 10⁹ or at least 10¹⁰ cfu/g, wherein the composition is frozen or freeze-dried.

10. The composition according to claims 8-9, further comprising lactic acid bacteria which are able to release glucose, preferably Streptococcus thermophilus and/or Lactobacillus delbrueckii subsp. bulgaricus.

11. A method of reducing free manganese in a product, preferably food or feed product, comprising:
selecting one or more lactobacillus strains comprising a manganese transporter MntH1, characterized in that the strain comprises inactivated MntR and/or inactivated binding site for MntR upstream of mntH1,
adding the strain(s) is said product to reduce free manganese, preferably as a Direct Vat Set (DVS) culture composition.

12. The method according to claim 11, wherein the product is a fermented food product, further comprising:
fermenting the product with the strain(s).

13. Use of the lactobacillus strain according to claims 5-7 or the composition according to claims 8-10 for reducing free manganese in products, preferably food products or fermented food products.

14. Use of the lactobacillus strain according to claims 5-7 or the composition according to claims 8-10 for inhibiting or delaying yeast, mold or listeria growth in products, preferably food products or fermented food products.

15. Use of the lactobacillus strain according to claims 5-7 or the composition according to claims 8-10 for preparing a fermented food product.

16. A method of producing a fermented dairy or dairy analogue products, comprising the steps of
a) adding a starter culture and the lactobacillus strain according to claims 5-7 or the composition according to claims 8-10 to a milk base,
b) fermenting the milk base for a period of time until a target pH, preferably 4.6, is reached.


SEQUENCE LISTING (71 sequences)

SEQ ID NO: 1 - protein, 535 aa, Lactobacillus paracasei
MSDDHKKRHPIKLIQYANGPSLEEINGTVEVPHGKGFWRTLFAYSGPGALVAVGYMDPGNWSTSITGGQNFQYLLISVILMSSLIAMLLQYMAAKLGIVSQMDLAQAIRARTSKKLGIVLWILTELAIMATDIAEVIGAAIALYLLFHIPLVIAVLVTVLDVLVLLLLTKIGFRKIEAIVVALILVILLVFVYQVALSDPNMGALLKGFIPTGETFASSPSINGMSPIQGALGIIGATVMPHNLYLHSAISQTRKIDHKNPDDVAQAVKFSAWDSNIQLSFAFVVNCLLLVMGVAVFKSGAVKDPSFFGLFQALSDSSTLSNGVLIAVAKSGILSILFAVALLASGQNSTITGTLTGQVIMEGFVHMKMPLWARRLVTRIISVIPVIVCVMLTARDTPIQQHEALNTLMNNSQVFLAFALPFSMLPLLMFTNSKVEMGDRFKNTGWVKVLGWISVLGLTGLNLKGLPDSIAGFFGDHPTATQTNMANIIAIVLIVAILALLAWTIWDLYKGNQRYEAHLAAVADEKEAKADVDEQ

SEQ ID NO: 2 - protein, 535 aa, Lactobacillus paracasei
MSDDHKKRHPIKLIQYANGPSLEEINGTVEVPHGKGFWRTLFAYSGPGALVAVGYMDPGNWSTSITGGQNFQYLLISVILMSSLIAMLLQYMAAKLGIVSQMDLAQAIRARTSKKLGIVLWILTELAIMATDIAEVIGAAIALYLLFHIPLVIAVLVTVLDVLVLLLLTKIGFRKIEAIVVALILVILLVFVYQVALSDPNMGALLKGFIPTGETFASSPSINGMSPIQGALGIIGATVMPHNLYLHSAISQTRKIDYKNPDDVAQAVKFSAWDSNIQLSFAFVVNCLLLVMGVAVFKSGAVKDPSFFGLFQALSDSSTLSNGVLIAVAKSGILSILFAVALLASGQNSTITGTLTGQVIMEGFVHMKMPLWARRLVTRIISVIPVIVCVMLTARDTPIQQHEALNTLMNNSQVFLAFALPFSMLPLLMFTNSKVEMGDRFKNTGWVKVLGWISVLGLTGLNLKGLPDSIAGFFGDHPTATQTNMANIIAIVLIVAILALLAWTIWDLYKGNQRYEAHLAAVADEKEAKADVDEQ

SEQ ID NO: 3 - protein, 535 aa, Lactobacillus casei
MSDDNKKKHSMKLIQYANGPSLEEINGTVEVPHGKGFWRTLFAYSGPGALVAVGYMDPGNWSTSITGGQNFQYLLISVILMSSLIAMLLQYMAAKLGIVSQMDLAQAIRARTSKTLGIVLWILTELAIMATDIAEVIGAAIALYLLFHIPLVISVLITVLDVLVLLLLTKIGFRKIEAIVVALILVILFVFIYQVALSDPNMGALLKGFIPTSKTFANSPSVNGMSPIQGALGIIGATVMPHNLYLHSAISQTRKIDHHDPDDVAQAVKFSAWDSNIQLSFAFVVNCLLLVMGVAVFKSGAVKDPSFFGLFEALSDSSTLSNGVLIAVAKSGILSILFAVALLASGQNSTITGTLTGQVIMEGFIHMKMPLWARRLVTRIISVIPVIVCVMLTARETPIQQHEALNTLMNNSQVFLAFALPFSMLPLLMFTNSKVEMGDRFKNTGWVKVLGWISVLGLTYLNLKGLPDSIAGFFGDHPTAAQTAIANDIAYALIVAVLALLAWTVWDLYKGNKRYEAHLEAVADAKEAKASNDVQ

SEQ ID NO: 4 - protein, 525 aa, Lactobacillus rhamnosus
MKLIQYANGPSLEEINGTVEVPHGKGFWRTLFAYSGPGALVAVGYMDPGNWSTSITGGQNFQYLLISVILMSSLIAMLLQYMAAKLGIVSQMDLAQAIRARTSKKLGIVLWILTELAIMATDIAEVIGAAIALYLLFHIPLVIAVLVTVLDVLVLLLLTKIGFRKIEAIVVALILVILLVFVYQVALSDPNMGALLKGFIPTGETFASSPSVNGMSPIQGALGIIGATVMPHNLYLHSAISQTRKIDHKDPEDVAQAVKFSAWDSNIQLTFAFVVNCLLLVMGVAVFKSGAVKDPSFFGLFQALSDSSTLSNGVLIAVAKSGILSILFAVALLASGQNSTITGTLTGQVIMEGFIHMKMPLWARRLVTRVISVIPVIVCVMLTARETPIQQHEALNTLMNNSQVFLAFALPFSMLPLLMFTNSKVEMGDRFKNTGWVKVLGWVSVIGLTYLNLKGLPDSIAGFFGDNPTAAQTNIANMIAYVLIAAVLALLAWTIWDLYKGNKRYEAHLEAVADEEEAKANDDVQ

SEQ ID NO: 5 - protein, 529 aa, Lactobacillus plantarum
SEKMNTPNRKHKLIEYANGPSLEEINGTIEVPKNLNFWKTLFAYSGPGALVAVGYMDPGNWSTSITGGQNYQYMLMSVILISSLIAMLLQYMAAKLGIVSQMDLAQAIRARTSKSLGIVLWILTELAIMATDIAEVIGAAIALYLLFNIPLVIAVFITVLDVLVLLLLTKIGFRKIEAIVVCLILVILFVFVYQVALSNPDWGGVIKGLVPTADTFSTSRSVNGMTPLSGALGIIGATVMPHNLYLHSAISQTRKIDHNDEEDVARTVKFAAWDSNIQLSFAFVVNSLLLIMGVAVFKSGAVKDPSFFGLYEALSNTSMLSNGILISVAKSGALSALFAIALLASGQNSTITGTLTGQVIMEGFVHMRMPLWLRRLVTRLISVIPVLICVLLTSGKSAIDEHTALNNLMNNSQVFLAFALPFSMLPLLMMTDSAAEMGKRFKNSLWIKGLGWLSVIGLTFLNLLGLPDSILGFFGDNPSAGEQTFSKILAYLLIAAILALLVWTVFDLQRGNKRYVEQQLAAAAKEANK

SEQ ID NO: 6 - protein, 521 aa, Lactobacillus salivarius
MVNNENNHKKHKMIQYANGKSLEEVNGTVEIPKGKGFWKTLFAYSGPGALVAVGYMDPGNWSTSITGGQNFQYLLMSVILLSSLIAMLLQYMAAKLGIVSQMDLAQAIRARTSKALGIVLWILTELAIMATDIAEVIGAAIALYLLFDIPLIIAVFITVFDVLLLLLLTKVGFRKIEAIVVCLIFVILFVFVYQVALSNPDWGGVFKGLIPTSETFAKHPVVHDMSPLNGALGIIGATVMPHNLYLHSAISQTRKFDRNNEDDIANAVRFTAWDSNIQLGLAFVVNSLLLIMGVAVFKSGAVEDPSFFGLYQALSDTSVMSNGLLAAAARTGILSTLFAVALLASGQNSTITGTLTGQVIMEGFIHLRMPLWARRLITRLLSVIPVLICVALTSGKSTIEEHEALNNLMNNSQVFLAFALPFSMLPLVIMTGSKVEMGERFKNRLWINILGWISVISLTYLNMIGLPQNLEPFFPADKVGLAHTVAYILIVLIIALLIWTLVELHLGNKRFAAEQAKKHNK

SEQ ID NO: 7 - protein, 526 aa, Lactobacillus fermentum
MRGGFGVDNTKNQHRKLRLIEHANGKSLEEINGTVEVPHGKGFFRTLFAYSGPGALVAVGYMDPGNWSTSITGGQSFQYTLMTTILISSLIAMLLQYMAAKLGIVSQMDLAQAIRARTGKALGVILWLMTELAIMATDIAEVIGAAIALNLLFHIPLVLAVFITVLDVLVLLLLTKIGFRKIEAIVACLILVILAVFAYQVALSHPDWAGVFKGLLPTKEAIAKEPVVGGISPLTGSLGIIGATVMPHNLYLHSAISQTRKIDHTNAEDIKQTVRFTAWDSNIQLTLAFFVNALLLIMGVAVFKNGAVQDSSFFGLYDALNNTDMLSNGLLIAVAKSGVLSTLFAIALLASGQNSTITGTLTGQVIMEGFVHMKMPLWARRLITRLLSVVPVLVCVAMTAHESTIDQHASLNILMENSQVFLAFALPFSMLPLLIMTNSDTEMGQFKNSLWVRVLGWISVIGLTFLNLYNLPQTYEGFGIWSKGLSDVLAWISIVVIVVLLAWTCFELIRGDRRLAAEREKHTWEK

SEQ ID NO: 8 - protein, 524 aa, Lactobacillus sakei
MNKVKGPKKHKLIEYANGPSLEEINGTVEVPEGKTFWKTLLAYSGPGALVAVGYMDPGNWSTSITGGQSFQYLLMSVILVSSLIAMLLQYMAAKLGIVTQMDLAQAIRARTSKSLGIVLWILTELAIMATDIAEVIGAAIALYLLFNIPLVIAVFITILDVMLLLLLTKVGFRKIEAIVVALIVVIFVVFAYEVALSNPDWAGVIVGLVPTAKTFATTPNVGGMSPLTGALGIIGATVMPHNLYLHSAISQTRKIDRNNEEQVAQTVRFSTWDSNIQLTMAFFVNALLLIMGVAVFKTGAVKDPSFFGLFEALSDTSTMSNGILASVARTGILSTLFAVALLASGQNSTITGTLTGQVIMEGFVHLRMPLWLRRLVTRLLSVIPVLICVMMTSNKPPLEEHQALNTLMNNSQVFLAFALPFSMLPLLMFTDSRVEMGDRFKNSLVIRVLGWLSVIGLTYLNMLGLPGQIEAFFGDHATAAQLALADHIAYVLIAAVLALLVWMIVELYKGNQRFEQQLAAQAAE

SEQ ID NO: 9 - protein, 546 aa, Lactobacillus reuteri
MISNLINKTQTKGGFGVDDTKNQHRKHKLIEYANGKSLEEINGTVEVPRGKGFWRTLFAYSGPGALVAVGYMDPGNWSTSITGGQSFQYTLMTTILISSLIAMLLQYMAAKLGIVSQMDLAQATRARTGKALGIILWIMTELAIMATDIAEVIGAAIALNLLFHIPLIPSVFITVLDVLVLLLLTKIGFRKIEAIVACLILVILFVFAYQVALSNPNWGGVFMGLLPSAKAIAQHPEIGGITPLTGTLGIIGATVMPHNLYLHSAISQTRKIDHNDLDSIRQTVRFTTWDSNIQLSLAFIVNSLLLIMGVAVFKTGAVQDSSFFGLYDALNNTSMLSNPVLIAVAKSGVLSTLFAVALLASGQNSTITGTLTGQVIMEGFIHMRMPLWARRLVTRIISVIPVIACVAMTSGENTIQQHTALNLLMENSQVFLAFALPFSMLPLLMMTNSEVEMGEFKNRGWVKVCGWISVIALTFLNLYNLPATYEGFGIWSKGTSDVLAYITIIVILALLIWTCVELYKGDKRFAAEGKGFGQREAQMKDSVVED

SEQ ID NO: 10 - protein, 533 aa, Lactobacillus brevis
MDLRKGVLKLSDNVQKKHKLISYANGRSLEEINGSVAVPKNISFWKALFMYSGPGALVAVGYMDPGNWSTSITGGQNFQYLLMSIILISSLIAMLLQYMAAKLGIVSQMDLAQAIRARTSKSLGIVLWIMTEFAIMATDIAEVIGAAIALYLLFHIPLVIAVFITVFDVLLLLLLTKIGFRKIEAIVVCLILVILVVFAYQVALSNPDWGGVFAGLIPSPKTIASTPQIGGQTPITGALGIIGATVMPHNLYLHSAISQTRQINHDDEEDVARTVRFSSWDSNIQLTLAFFVNALLLIMGVAVFKSGAVKDPSFFGLFQALSDTNTMSNGVLAGVAKTGALSTLFAVALLASGQNSTITGTLTGQVIMEGFVHMRMPLWLRRLVTRLISVIPVLICVMMTSGKSAIDEHTALNDLMNNSQVFLAFALPFSMLPLLMMTDSKLEMGERFKNSAWVKWLGWLSVLTLTGLNLYNMPASIQGFYGDGITASETMTADVIAWVLNAAIIALLVWTIYELRKGNRRLAQAVAADGKTN

SEQ ID NO: 11 - protein, 533 aa, Lactobacillus kefiri
MPKEKQPKKQHLIHYANGPSLEEINGTIEVPKGRGFWKTLFMYSGPGALVAVGYMDPGNWSTSITGGQNFEYLLMSVILLSSLIAMLLQYMAAKLGIVSQMDLAQAIRARTSKTLGIVLWILTELAIMATDIAEVIGAAIALYLLFHIPLVYAVFITVFDVLLLLLLTKVGFRKIEAIVVCLILVILFVFIYEVALSEPDFGAMVKGLIPTGQTFSSADHVNGDTPLTGALGIIGATVMPHNLYLHSAISQTRKVDHNDQDDVARTVRFSTWDSNIQLSFAWVINSLLLVMGVAVFKTGAVKDPSFFGLFDALSNTSMLSNGILIAVAKSGILSILFAVALLASGQNSTITGTLTGQVIMEGFIHMKMPLWLRRLVTRLISVIPVLICVAMTSNETPIKQHEALNTLMNNSQVFLAFALPFSMLPLLMMTDNAKEMGERFKNTLWVKVLGWVSVLALTFLNMKGLPDNITSFFGAAPSASQVSLAHTIAYVIIVAIVLLLLWTVYDLYSSRNKMPQRFETTAEHYDESKKDKE

SEQ ID NO: 12 - protein, 551 aa, Lactobacillus alimentarius
MAEKHKLIEYANGPSLQEINGTVDVPKGKGFFKTLFAYSGPGALVAVGYMDPGNWSTSITGGQNFQYLLMSVILMSSLIAMLLQYMAAKLGIVSKMDLAQAIRARTSRSLGIVLWILTELAIMATDIAEVIGGAIALYLLFNIPLVIAVFITVGDVLVLLLLTKIGFRKIEAIVVCLILVILFVFVYQVALSNPDWGGVFAGLIPTGKTFATGPKIGGQTPLNGALGIIGATVMPHNLYLHSAISQTRKVDHADEASVAQNVRFSAWDSNIQLTAAFFVNALLLIMGVAVFKSGAVEDPSFFGLYKALSDTSTLSNGVLIAVAKSGILSTLFAVALLASGQNSTITGTLTGQVIMEGFVHMRMPLWLRRLVTRLISVIPVLICVMLTSGKSAIDEHEALNTLMNNSQVFLAFALPFSMLPLLLMTDSATEMGNKFKNAAWIKIFGWLSVIALTFLNLYGLPDQIKAFYGDGITSAQSLQANIIAYVLIAAVLALLVWTVFDMHKGNERLKTVLAKEDVTSTYEHLAKISASVSSEEDFDKQATAERNSEQR

SEQ ID NO: 13 - protein, 522 aa, Lactobacillus curvatus
MNQQEKGKKHKLIEYANGPSLEEINGTVEVPEGKGFWKMLLAYSGPGALVAVGYMDPGNWSTSITGGQSFQYLLMSVILVSSLIAMLLQYMAAKLGIVTQMDLAQAIRARTSKPLGIVLWILTELAIMATDIAEVIGAAIALYLLFKIPLLIAVFITILDVMLLLLLTKIGFRKIEAIVVALIVVIFVVFAYEVALSDPDWAGVIVGLVPTAKTFATGPAVGGLTPLTGALGIIGATVMPHNLYLHSAISQTRKIDRKNEAQVAQTVRFATWDSNIQLTMAFFVNALLLIMGVAVFKTGTVKDPSFFGLFKALSDTSTMSNGILASVARTGILSTLFAVALLASGQNSTITGTLTGQVIMEGFIHLRMPLWLRRLVTRLLSVIPVLICVMMTSNKPALEEHEALNTLMNNSQVFLAFALPFSMLPLLMFTDSRVDMGDRFKNSWLIKSLGWLSVIGLTYLNMMGLPGQIEAFYGDHASAAQLATADRIAYVLIAGVMALLVWMIIELYKGNKRFEQQLATEN

SEQ ID NO: 14 - protein, 535 aa, Lactobacillus zeae
MSDDHKKKHSMKLIQYANGPSLEEINGTVEVPHGKGFWRTLFAYSGPGALVAVGYMDPGNWSTSITGGQNFQYLLISVILMSSLIAMLLQYMAAKLGIVSQMDLAQAIRARTSKTLGIVLWILTELAIMATDIAEVIGAAIALYLLFHIPLVISVLITVLDVLVLLLLTKIGFRKIEAIVVALILVILFVFIYQVALSDPNMGALLKGFIPTSETFANSPSVNGMSPIQGALGIIGATVMPHNLYLHSAISQTRKIDHHDPDDVAQAVKFSAWDSNIQLSFAFVVNCLLLVMGVAVFKSGAVKDPSFFGLFEALSDSSTLSNGVLIAVAKSGILSILFAVALLASGQNSTITGTLTGQVIMEGFIHMKMPLWARRLVTRIISVIPVIVCVMLTARETPIQQHEALNTLMNNSQVFLAFALPFSMLPLLMFTNSKVEMGERFKNTGWVKVLGWISVLGLTYLNLKGLPDSIAGFFGDHPTATQTTIANDIAYALIVAVLALLAWTIWDLYKGNKRYEAHMEAVADAKEAKASNDVQ

SEQ ID NO: 15 - protein, 529 aa, Lactobacillus kimchicus
MKDQNTPRKHHLIEYANGKSLEEINGTVEVPKGRGFWRTLFMYSGPGALVAVGYMDPGNWSTSITGGQNFQYLLMSVILMSSLIAMLLQYMAAKLGIVSQMDLAQAIRARTSKSLGVVLWILTELAIMATDIAEVIGAAIALYLLFHIPLVYAVFITVFDVLLLLLLTKVGFRKIEAIVVCLILVILFVFVYQVALSNPNWAAVIGGLVPTGETFSSSPSVGGMTPISGALGIIGATVMPHNLYLHSAVSQSRKIDHNDEEDVARTVRFSTWDSNIQLSFAFVVNSLLLIMGVAVFKTGAVKDPSFFGLFEALSNTSTLSNGVLIGVAKSGVLSVLFAVALLASGQNSTITGTLTGQVIMEGFVHMRMPLWLRRLVTRLISVIPVLICVAITGRETPIQQHEALNNLMNNSQVFLAFALPFSMLPLLMMTNSRLEMGQRFKNNFLVKLFGWISVIALTFLNMKGLPGSIAGFYGDNITAAQTHQANIIAYILIAAVLALLVWTVYDLYKGNQRLAAKLAAEPSNNDVAD

SEQ ID NO: 16 - DNA, 19 bp, Lactobacillus sp.
  source 1..19: mol_type = other DNA
dddkwwrsknnnchwamma

SEQ ID NO: 17 - DNA, 20 bp, Lactobacillus brevis
  source 1..20: mol_type = genomic DNA
atttttggtaagccaaaaat

SEQ ID NO: 18 - DNA, 20 bp, Lactobacillus brevis
  source 1..20: mol_type = genomic DNA
aaggaagggagtcttaaaat

SEQ ID NO: 19 - DNA, 20 bp, Lactobacillus casei
  source 1..20: mol_type = genomic DNA
gaattaggtcaccctaaaaa

SEQ ID NO: 20 - DNA, 20 bp, Lactobacillus casei
  source 1..20: mol_type = genomic DNA
aagttagggagacctaaaag

SEQ ID NO: 21 - DNA, 20 bp, Lactobacillus fermentum
  source 1..20: mol_type = genomic DNA
tttttatgctaacctaacaa

SEQ ID NO: 22 - DNA, 20 bp, Lactobacillus plantarum
  source 1..20: mol_type = genomic DNA
aagttaactgcacctaacaa

SEQ ID NO: 23 - DNA, 20 bp, Lactobacillus plantarum
  source 1..20: mol_type = genomic DNA
tttgtaggcatacctaaaaa

SEQ ID NO: 24 - DNA, 20 bp, Lactobacillus reuteri
  source 1..20: mol_type = genomic DNA
tttttatgttaccctaacaa

SEQ ID NO: 25 - DNA, 20 bp, Lactobacillus rhamnosus
  source 1..20: mol_type = genomic DNA
aagttagggagacctaaaag

SEQ ID NO: 26 - DNA, 20 bp, Lactobacillus rhamnosus
  source 1..20: mol_type = genomic DNA
gaattaggtcaccctaaaat

SEQ ID NO: 27 - DNA, 20 bp, Lactobacillus sakei
  source 1..20: mol_type = genomic DNA
aagttaggtatacctaaaag

SEQ ID NO: 28 - DNA, 20 bp, Lactobacillus sakei
  source 1..20: mol_type = genomic DNA
aagttagggcatcctaaaat

SEQ ID NO: 29 - DNA, 20 bp, Lactobacillus sakei
  source 1..20: mol_type = genomic DNA
aagttaagggacccaaaaag

SEQ ID NO: 30 - DNA, 20 bp, Lactobacillus salivarius
  source 1..20: mol_type = genomic DNA
agttaaggtagacctaaaaa

SEQ ID NO: 31 - protein, 214 aa, Lactobacillus paracasei
MTPNKEDYLKLIFEIGGDTQLVSNKQIVAGMHVSAASVSEMINKLGEEKLVAHTPYQGIQLTSAGRKKAAILVRNHRLWEVFLVQCLKYPADAVHQEAEKLEHALTPEMAKRLSAMLGEPRYCPHGGVIPDANGHYLQQSRVTLGTLDVGQSGHIERVIDEVSLIDYTVKIDLRLDDEFTVTAKTLDAVIIKLARTGKELAVDADRADHIFVEL

SEQ ID NO: 32 - protein, 214 aa, Lactobacillus casei
MTPNKEDYLKLIFEIGGDTELVSNKQIVAGMHVSAASVSEMINKLGEEKLVAHTPYQGIQLTSAGRKKAAILVRNHRLWEVFLVKCLKYSPDAVHQEAEKLEHALTPEMAKRLSAMLGNPEYCPHGGVIPDAEGHYIQQSRVTLGTMEVGQRGHIERVIDEVSLIDYTVKIDLRLDDAFTVTAKTLDAVIIKLDRTGKELAVDADRAAHIFVEL

SEQ ID NO: 33 - protein, 214 aa, Lactobacillus rhamnosus
MTPNKEDYLKLIFEIGGDTELVSNKQIVAGMHVSAASVSEMINKLGEEKLVAHTPYQGIQLTSAGRKKAAILVRNHRLWEVFLVKCLKYPADAVHQEAEKLEHALTPEMAKRLAAMLGNPQYCPHGGVIPDADGHYIQQSRVTLGAMEVGQKGHIERLIDEVSLIDYTVKLDLRLDDVFTVTAKTLDAVVIKLDRTGKELAVDADRAAHIFVEL

SEQ ID NO: 34 - protein, 215 aa, Lactobacillus plantarum
MTPMKEDYLKIIFELGGTKKKVSNKQIALSLDIAAGSVTEMVGKLVQEGLAKHTPYAGISLTKKGIRYAETLVRKHRIWEDFLVDKLDYDLPDVHTEAEVLEHVTSERLVDSLEAFLGNPTHCPHGGAIPDKDGHYQEDSHVSLADTQDGTTVTIERFIDNHDLLVYLHDTPLKIGQQVTVLKHDPFEGPVTVSIQKTGEEIPVSYKAAHNVFVK

SEQ ID NO: 35 - protein, 215 aa, Lactobacillus salivarius
MTPKKEDYLKIIFELGGTKKKVSNKQIAMSLNVAAGSVTEMVNKLVKEGLAAHTPYAGISLTDEGIEVAEKLVRRHRLWETFLVEKLDYQLSEVHDEAEVLEHVASDKLMKKLDQFLNSPRECPHGGVIPTEAGEYEEESHEYLAEIKVGETVEVDRFIDNHELLTYLDDLELKLGDKIEVLEHLPFEGPIKVKRLADGAELSIGYKAAHYIFVK

SEQ ID NO: 36 - protein, 219 aa, Lactobacillus fermentum
MTPMKEDYLKIIFELGGGRKKVSNKEISLGLGIAAGSVTEMITKLADEGLVEHEPYAGIALTEKGGRYAAELVRKHRLWETFLVDKLHYNMTDVHPEAEILEHKTSDHLATALDDFLGHPAYCPHGGVIPSANGRFTNISHRLLAEGEDGEEVIIERFLDNHDLLTYLSEIGLRLRDYIKIVKHEPFEGPVVVERLTDGQTLNISYKAAHNVFITPKDK

SEQ ID NO: 37 - protein, 220 aa, Lactobacillus sakei
MTPNKEDYLKIIFELGGDAKKVTNKEILAGLNVSAASVTEMVNKLVKENYVNHTPYQGIQLTSEGAREAALLVRNHRLWEVFLVDKLHYQFNTVHPEAEQLEHVTNHDLAERLADFLGHPKRCPHGGIIPNAKGEFEQQSHHALVDLEVGEKAVIERVLDDNDLLKYTLEIGLTVGDTVTLTKVGLFESPITVMDETQQTEIQVGIKAAQHIFVTPIAAD

SEQ ID NO: 38 - protein, 224 aa, Lactobacillus reuteri
MTPMKEDYLKIIFELGGSHKKVSNKEISLGLGIAAGSVTEMISKLADEGLVVHEPYAGISLTEKGQKYAAELVRKHRLWETFLVDKLHYNFADVHSEAEILEHQTSDRLATALDSFLQHPDHCPHGGVIPSANGKFPDVTHRLLADADDGEKVELERFLDNHELLTYLEELGLRPQEQVTVIRHEPFEGPIVIQKENNDQEINVSYKASHNIFIEPDTAQENKD

SEQ ID NO: 39 - protein, 215 aa, Lactobacillus brevis
MTPMKEDYLKIIFELGGRQKKVSNKQIAISLNIAAGSVTEMVNKMAAEGLAEHTPYAGISLTNRGIRLAEDLVRKHRIWEDFLVEKLGYALPDVHDEAEVLEHVTSPKLIDALDDMLGNPTHCPHGGVIPDRQGHYHEDSHTVLNDAADGEIVTVDRFIDNHDLLTYLGDLKLDIGDQLQVLKHDPFEGPVTVQNLTDNAELIVSYKAAHYIFVK

SEQ ID NO: 40 - protein, 215 aa, Lactobacillus kefiri
MTPMKEDYLKIIFELGGSDDLVSNKQIAISLNIAAGSVTEMVNKLVEEKLVTHEPYSGVQLTKKGKKYAEELVRKHRIWETFLANTLHYDISDVHDEAELLEHVTSDKMIDHLDDFLGNPKRCPHGGVIPDRNGNYHPDKDKLLTDAKDGEEVVVNRFIDNHDLLTLLGDIKLDIGDKLKIISHDPFEGSVTVKNLTDKKKLVIGFKTAHYVFVR

SEQ ID NO: 41 - protein, 216 aa, Lactobacillus alimentarius
MSPNKENYLKTIYELNYDFTKITNKRISEIMNVSAPSVTEMLNSLSSEGYLTHTPYNKIVLTPKGNKVSEKLVRTHRLWEVFLHECLKYPVDNVHHNADALEHASDDGLIDHLNDFLDHPQRCPHGGIIPGNGQGETDADDKLLSMIPDNTKVQIVRVSDNYDFLQYFGSLNLEIDDTIEVLKHEKFDNSLVVKKEDGTKLTIGAKAIDYIFVELR

SEQ ID NO: 42 - protein, 218 aa, Lactobacillus curvatus
MTPNKEDYLKIIFELGGDAKKVTNKEILAGLNVSAASVTEMVNKLVKENYVNHTPYQGIQLTSEGAREAALLVRNHRLWEVFLVDKLHYQFNTVHPEAEQLEHVTNHDLAERLADFLGHPTRCPHGGIIPNAKGEFEQQSHIALETLQVGETAIIDRVLDDNDLLKYTLEIGLSVGDSVTLQKVGLFESPLTVFNNTSQTEIQIGLKAAQHIFVTPQN

SEQ ID NO: 43 - protein, 214 aa, Lactobacillus zeae
MTPNKEDYLKLIFEIGGDTELVSNKQIVAGMHVSAASVSEMINKLGEEKLVAHTPYQGIQLTSAGRKKAAILVRNHRLWEVFLVKCLKYSPDAVHQEAEKLEHALTPEMAKRLSAMLGNPEYCPHGGVIPDAEGHYIQQSRVTLGAMEVGQRGHIERLIDEVSLIDYTVKIDLRLDDAFTVTAKTLDAVVIKLDRTGKELAVDADRAAHIFVEL

SEQ ID NO: 44 - protein, 215 aa, Lactobacillus kimchicus
MTPMKEDYLKLIFEIGGGSQKVSNKQIAISLDIAAGSVTEMVTKMAAEGLVEHEPYAGISLTETGAKLAVELVRKHRIWETFLVSELKYALPDIDDDAEKLEHVTSTKLLNALDDLLGHPKRCPHGGVIPDRNGHYEEDSHRILNDVKDGETVVVDRFIDNRDLLNYLGDIKLDLGDQLQVIKHDSFEGPILVENLTDDSELSIGYKAAHYIFVK

SEQ ID NO: 45 - DNA, 25 bp, Synthetic construct
  source 1..25: mol_type = other DNA
gcttatcgataccgtcgacctcgag

SEQ ID NO: 46 - DNA, 50 bp, Synthetic construct
  source 1..50: mol_type = other DNA
gtcgttaaatgccctttacctgttccaatttcgtaaacggtatcggtttc

SEQ ID NO: 47 - DNA, 50 bp, Synthetic construct
  source 1..50: mol_type = other DNA
gaaaccgataccgtttacgaaattggaacaggtaaagggcatttaacgac

SEQ ID NO: 48 - DNA, 27 bp, Synthetic construct
  source 1..27: mol_type = other DNA
cgggggatccactagttctagagcggc

SEQ ID NO: 49 - DNA, 50 bp, Synthetic construct
  source 1..50: mol_type = other DNA
gccgctctagaactagtggatcccccggaatctgaataagacaaagcttg

SEQ ID NO: 50 - DNA, 44 bp, Synthetic construct
  source 1..44: mol_type = other DNA
ccctgctttctctagacttaatttacatccttacttttaatttg

SEQ ID NO: 51 - DNA, 44 bp, Synthetic construct
  source 1..44: mol_type = other DNA
caaattaaaagtaaggatgtaaattaagtctagagaaagcaggg

SEQ ID NO: 52 - DNA, 48 bp, Synthetic construct
  source 1..48: mol_type = other DNA
ctcgaggtcgacggtatcgataagccataattttgcctgcgacaaaag

SEQ ID NO: 53 - DNA, 52 bp, Synthetic construct
  source 1..52: mol_type = other DNA
gaagaaggtttttatattacagctccagatctagcgctatagttgttgacag

SEQ ID NO: 54 - DNA, 52 bp, Synthetic construct
  source 1..52: mol_type = other DNA
cttggttttctaattttggttcaaagaaagcttttatttgtacagctcatcc

SEQ ID NO: 55 - DNA, 52 bp, Synthetic construct
  source 1..52: mol_type = other DNA
ggatgagctgtacaaataaaagctttctttgaaccaaaattagaaaaccaag

SEQ ID NO: 56 - DNA, 52 bp, Synthetic construct
  source 1..52: mol_type = other DNA
ctgtcaacaactatagcgctagatctggagctgtaatataaaaaccttcttc

SEQ ID NO: 57 - DNA, 22 bp, Synthetic construct
  source 1..22: mol_type = other DNA
tctagagtgagtaaaggcgaag

SEQ ID NO: 58 - DNA, 26 bp, Synthetic construct
  source 1..26: mol_type = other DNA
agatctggagctgtaatataaaaacc

SEQ ID NO: 59 - DNA, 42 bp, Synthetic construct
  source 1..42: mol_type = other DNA
tcttcttcgcctttactcacaataactctcccctttcgtttg

SEQ ID NO: 60 - DNA, 46 bp, Synthetic construct
  source 1..46: mol_type = other DNA
ggtttttatattacagctccagatctgtgactttttaacaataacg

SEQ ID NO: 61 - DNA, 31 bp, Synthetic construct
  source 1..31: mol_type = other DNA
agtggatcccccgccgaagctgaataagacc

SEQ ID NO: 62 - DNA, 36 bp, Synthetic construct
  source 1..36: mol_type = other DNA
cccaagtcttgatttacatccttacttttaatttgc

SEQ ID NO: 63 - DNA, 32 bp, Synthetic construct
  source 1..32: mol_type = other DNA
gtaaggatgtaaatcaagacttgggaaagcag

SEQ ID NO: 64 - DNA, 35 bp, Synthetic construct
  source 1..35: mol_type = other DNA
ggtatcgataagcaaaatcttgcctgcgacaaaag

SEQ ID NO: 65 - DNA, 31 bp, Synthetic construct
  source 1..31: mol_type = other DNA
aggcaagattttgcttatcgataccgtcgac

SEQ ID NO: 66 - DNA, 30 bp, Synthetic construct
  source 1..30: mol_type = other DNA
attcagcttcggcgggggatccactagttc

SEQ ID NO: 67 - DNA, 31 bp, Synthetic construct
  source 1..31: mol_type = other DNA
aagtgagggaaaggctactaaaacgtcgagg

SEQ ID NO: 68 - DNA, 60 bp, Synthetic construct
  source 1..60: mol_type = other DNA
ttataatccatggttcattgattgattacactgtatctacaagagtagaaattaaaaagg

SEQ ID NO: 69 - DNA, 57 bp, Synthetic construct
  source 1..57: mol_type = other DNA
tactcttgtagatacagtgtaatcaatcaatgaaccatggattataaagagagcggc

SEQ ID NO: 70 - DNA, 30 bp, Synthetic construct
  source 1..30: mol_type = other DNA
cgttttagtagcctttccctcacttcgttc

SEQ ID NO: 71 - DNA, 21 bp, Synthetic construct
  source 1..21: mol_type = other DNA
acagtgtaatcaatcaatgaa